(19) United States
(12) Patent Application Publication
(10) Pub. No.: US 2026/0001840 A1
(43) Pub. Date: Jan. 1, 2026

(54) Functionalized Conjugation Crosslinkers and Uses Thereof

(71) Applicant: Azvlz, Inc., San Diego, CA (US)

(72) Inventors: Ying Zeng, San Diego, CA (US); Chudai Zeng, San Diego, CA (US); Lijun Liu, San Diego, CA (US)

(21) Appl. No.: 19/102,683

(22) PCT Filed: Jan. 6, 2025
(86) PCT No.: PCT/US2025/010493

§ 371 Date: Feb. 10, 2025

Related U.S. Application Data
(60) Provisional application No. 63/618,705, filed on Jan. 8, 2024.

(51) Int. Cl. C07D 207/46; C07D 403/12

(52) U.S. Cl. CPC C07D 207/46; C07D 403/12

(57) ABSTRACT

The present invention relates to multi-functionalized crosslinker compounds with a protected aminooxy group and a N-hydroxysuccinimide ester (NHS) activated carbonyl group. The crosslinkers may form conjugates of biomolecules (e.g., antibodies, oligonucleotides, lipids) with each other or with small molecules. The resulting conjugates may have applications in various therapeutic areas, such as, for example, cancers, infectious disease autoimmune disorders, neurodegenerative disease, etc., diagnostic areas and drug delivery. The crosslinkers may also be used to functionalize the surface of various materials such as, for example, metals, nanoparticles, etc. with biological molecules and small molecules, which may be useful in various diagnostic, therapeutic and drug delivery technologies.

### CROSS REFERENCE TO RELATED APPLICATIONS

This application is a national stage 371 application of PCT/US2025/010493, filed on Jan. 6, 2025, which claims priority to U.S. Provisional Application Ser. No. 63/618,705 filed Jan. 8, 2024, under 35 U.S.C. § 119(e) which is herein incorporated by reference in its entirety for all purposes.

### TECHNICAL FIELD

The present invention relates to multi-functionalized crosslinker compounds with a protected aminooxy group and a N-hydroxysuccinimide ester (NHS) activated carbonyl group. The crosslinkers may form conjugates of biomolecules (e.g., antibodies, oligonucleotides, lipids) with each other or with small molecules. The resulting conjugates may have applications in various therapeutic areas, such as, for example, cancers, infectious disease autoimmune disorders, neurodegenerative disease, etc., diagnostic areas and drug delivery. The crosslinkers may also be used to functionalize the surface of various materials such as, for example, metals, nanoparticles, etc. with biological molecules and small molecules, which may be useful in various diagnostic, therapeutic and drug delivery technologies.

### BACKGROUND

Multi-functionalized crosslinker compounds are used to facilitate the attachment of biomolecules (e.g., antibodies, oligonucleotides, lipids) with each other or with small molecules. Multi-functionalized crosslinker compounds are essential in bioconjugation reactions which significantly expands the scope and medical applications of various biomolecules, in therapy, drug delivery, diagnostics, and the study of biological systems.

Aldehydes are useful agents for the covalent attachment of biomolecules. Aldehydes can be introduced onto various molecules, such as proteins, oligonucleotides, and carbohydrates, through periodate oxidation, and introduction of unnatural amino acids or aldehyde-containing reagents. The resulting aldehyde can be selectively reacted with aminooxy compounds in aqueous solution to form a stable covalent oxime bond. Aminooxy group consists of an amino group attached to an oxygen atom and are highly useful in bioconjugation due to their ability to react with carbonyl compounds specifically and selectively, such as aldehydes and ketones, to form stable oxime bonds.

N-hydroxy succinimide (NHS) esters are widely used in bioconjugation chemistry and can be selectively reacted with primary amine groups to form stable amide bonds. Accordingly, NHS esters are also selectively reactive with aminooxy group which also includes a primary amine group.

A significant challenge in bioconjugation chemistry is having crosslinkers with both a NHS ester and an aminooxy group because of the cross reactivity between the two functional groups. Accordingly, typical amine protecting groups such as Fmoc, t-Boc, Troc, phthalimide or trifluoroacetamide groups were used to mask the reactivity of the aminooxy group. However, harsh reaction conditions are necessary to remove these amine protecting groups, which severely limited the use of crosslinkers which include a NHS ester and an aminooxy group under physiological conditions.

Accordingly, there is a need for an amine protecting group which can be removed under mild conditions and hence enhance the utility of crosslinkers which include a NHS ester and an aminooxy group to provide bioconjugates with a stable oxime linkage.

### SUMMARY

The instant invention satisfies these and other needs by providing in one aspect, a compound of formula (I):

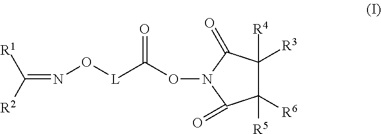

or salts, hydrate or solvates thereof wherein: R¹ and R² are independently alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, heterocycloalkyl, substituted heterocycloalkyl, heteroalkyl, substituted heteroalkyl, heteroalkenyl, substituted heteroalkenyl, heteroaryl, substituted heteroaryl, heteroarylalkyl, substituted heteroarylalkyl, heteroarylalkenyl, substituted heteroarylalkenyl, or together with the atoms to which they are bonded form a cycloalkyl, substituted cycloalkyl, cycloalkenyl, substituted cycloalkenyl, heterocycloalkyl, substituted heterocycloalkyl, heterocycloalkenyl or substituted heterocycloalkenyl ring; R³, R⁴, R⁵ and R⁶ are independently —H, alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, heterocycloalkyl, substituted heterocycloalkyl, heteroalkyl, substituted heteroalkyl, heteroalkenyl, substituted heteroalkenyl, heteroaryl, substituted heteroaryl, heteroarylalkyl, substituted heteroarylalkyl, heteroarylalkenyl, substituted heteroarylalkenyl or optionally one of R³, R⁴, R⁵ and R⁶ is —SO₃H or a salt thereof; L is alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl, substituted heteroarylalkenyldiyl, a complementary oligonucleotide between 1 and 30 nucleic acid bases, a complementary oligopeptide between 1 and 30 amino acids or -L₁X(L₂Z)ₐL₃-; L₁, L₂ and L₃ are independently alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl or substituted heteroarylalkenyldiyl; X is alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl, substituted heteroarylalkenyldiyl, alkyltriyl, heteroalkyltriyl, substituted heteroalkyltriyl, a complementary oligopeptide between 1 and 30 amino acids, a complementary oligonucleotide between 1 and 30 nucleic acid bases, —(CH₂)ᵣ—S(CH₂)ᵣ—, —(CH₂)ᵣ(OCH₂CH₂)ₛ(CH₂)ᵣ—, —(CH₂)ᵣC(O)(CH₂)ᵣ—, —(CH₂)ᵣC(O)₂(CH₂)ᵣ—, —(CH₂)ᵣS(CH₂)ᵣ— or (CH₂)ᵣSO₂(CH₂)ᵣ—; each r is independently an integer between 3 and 25; Z is maleimide, Texas Red, Alexa Fluor 48, Fluorescein isothiocyanate, Pyridine disulfide, Rhodamine, Rhodamine B, Tetramethyl Rhodamine, N₃, alkyne,

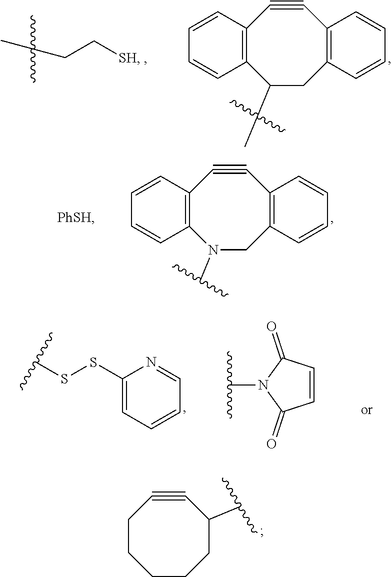

and a is 0 or 1.

In another aspect, crosslinkers are used to form conjugates of biomolecules (e.g., antibodies, oligonucleotides, lipids, proteins, etc.) with each other or with small molecules. Crosslinkers may also be used to functionalize the surface of various materials such as, for example, metals, nanoparticles, etc. with biological molecules and small molecules. The resulting conjugates may have applications in various therapeutic areas, such as, for example, cancers, infectious disease autoimmune disorders, neurodegenerative disease, etc., diagnostic areas and drug delivery.

### DETAILED DESCRIPTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used in the specification and the appended claims, the indefinite articles “a” and “an” and the definite article “the” can include plural referents as well as singular referents unless specifically stated otherwise or the context clearly indicates otherwise. The term “exemplary” as used herein means “serving as an example, instance or illustration”. Any embodiment or feature characterized herein as “exemplary” is not necessarily to be construed as preferred or advantageous over other embodiments or features.

Where elements are presented in list format (e.g., in a Markush group), it is understood that each possible subgroup of the elements is also disclosed, and any one or more elements can be removed from the list or group.

It is further understood that the disclosure of a numerical range is a specific disclosure of all the possible subranges and all the possible individual numbers (whether whole numbers or fractions) within that range regardless of the breadth of that range. It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment or compound found within the prior art, can be explicitly excluded from the claims, regardless of whether or not the specific exclusion is recited in the specification.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. It should be understood that u to v carbons includes u+1 to v, u+2 to v, u+3 to v, etc. carbons, u+1 to u+3 to v, u+1 to u+4 to v, u+2 to u+4 to v, etc. and cover all possible permutation of u and v.

“Alkyl,” by itself or as part of another substituent, refers to a saturated, branched, or straight-chain monovalent hydrocarbon radical derived by the removal of one hydrogen atom from a single carbon atom of a parent alkane. Typical alkyl groups include, but are not limited to, methyl; ethyl; propyls such as propan-1-yl, propan-2-yl, etc.; butyls such as butan-1-yl, butan-2-yl, 2-methyl-propan-1-yl, 2-methyl-propan-2-yl, etc.; and the like. In some aspects, an alkyl group comprises from 1 to 20 carbon atoms (C₁-C₂₀ alkyl). In other aspects, an alkyl group comprises from 1 to 10 carbon atoms (C₁-C₁₀ alkyl). In still other aspects, an alkyl group comprises from 1 to 6 carbon atoms (C₁-C₆ alkyl).

Alkyldiyl” by itself or as part of another substituent, refers to a saturated, branched or straight-chain hydrocarbon group derived by the removal of one hydrogen atom from each of two different carbon atoms of a parent alkane, or by the removal of two hydrogen atoms from a single carbon atom of a parent alkane. The two monovalent radical centers or each valency of the divalent radical center can form bonds with the same or different atoms. Typical alkyldiyl groups include, but are not limited to methandiyl; ethyldiyls such as ethan-1,1-diyl, ethan-1,2-diyl; propyldiyls such as propan-1,1-diyl, propan-1,2-diyl, propan-2,2-diyl, propan-1,3-diyl, butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, etc.; and the like. In some embodiments, the alkyldiyl group is (C₁-C₂₀) alkyldiyl. In other embodiments, the alkyldiyl group is (C₁-C₁₀) alkyldiyl. In still other embodiments, the alkyldiyl group is (C₁-C₆) alkyldiyl.

Alkyltriyl” by itself or as part of another substituent, refers to a saturated, branched or straight-chain hydrocarbon group derived by the removal of three hydrogen atom from carbon atom of a parent alkane. The three monovalent radical centers can form bonds with the same or different atoms. In some embodiments, the alkyltriyl group is (C₁-C₂₀) alkyltriyl. In other embodiments, the alkyltriyl group is (C₁-C₁₀) alkyltriyl. In still other embodiments, the alkyltriyl group is (C₁-C₆) alkyltriyl.

“Alkenyl,” by itself or as part of another substituent, refers to an unsaturated branched, straight-chain having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, etc.; and the like. In some aspects, an alkenyl group comprises from 2 to 20 carbon atoms (C₂-C₂₀ alkenyl). In other aspects, an alkenyl group comprises from 2 to 10 carbon atoms (C₂-C₁₀ alkenyl). In still other aspects, an alkenyl group comprises from 2 to 6 carbon atoms (C₂-C₆ alkenyl).

Alkenyldiyl” by itself or as part of another substituent, refers to a unsaturated, branched, straight-chain or cyclic divalent hydrocarbon group derived by the removal of one hydrogen atom from each of two different carbon atoms of a parent alkene, or by the removal of two hydrogen atoms from a single carbon atom of a parent alkene. The two monovalent radical centers or each valency of the divalent radical center can form bonds with the same or different atoms. Typical alkenyldiyl groups include, but are not limited to ethen-1,1-diyl, prop-1-en-1,1-diyl, prop-1-en-1,2-diyl, butyldiyls such as, but-1-en-1,1-diyl, but-1-en-1,2-diyl, but-1-en-1,3-diyl, but-1-en-1,4-diyl, 2-methyl-prop-1-en-1,1-diyl, etc.; and the like. In some embodiments, the alkenyldiyl group is (C₂-C₂₀) alkenyldiyl. In other embodiments, the alkenyldiyl group is (C₂-C₁₀) alkenyldiyl. In still other embodiments, the alkenyldiyl group is (C₂-C₆) alkenyldiyl.

“Alkynyl,” by itself or as part of another substituent refers to an unsaturated branched, straight-chain having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some aspects, an alkynyl group comprises from 2 to 20 carbon atoms (C₂-C₂₀ alkynyl). In other aspects, an alkynyl group comprises from 2 to 10 carbon atoms (C₂-C₁₀ alkynyl). In still other aspects, an alkynyl group comprises from 2 to 6 carbon atoms (C₂-C₆ alkynyl).

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In some aspects, an aryl group comprises from 6 to 30 carbon atoms (C₆-C₃₀ aryl). In other aspects, an aryl group comprises from 6 to 20 carbon atoms (C₆-C₂₀ aryl). In still other aspects, an aryl group comprises from 6 to 15 carbon atoms (C₆-C₁₅ aryl). In still other aspects, an aryl group comprises from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 1-phenylethen-1-yl, naphthylmethyl, 2-naphthylethan-1-yl, 1-naphthylethen-1-yl, naphthobenzyl, 2-naphthophenylethan-1-yl and the like. In some aspects, an arylalkyl group is (C₇-C₄₀) arylalkyl, e.g., the alkyl moiety of the arylalkyl group is (C₁-C₁₀) alkyl and the aryl moiety is (C₆-C₃₀) aryl. In other aspects, an arylalkyl group is (C₇-C₃₀) arylalkyl, e.g., the alkyl moiety of the arylalkyl group is (C₁-C₁₀) alkyl and the aryl moiety is (C₆-C₂₀) aryl. In other aspects, an arylalkyl group is (C₇-C₂₀) arylalkyl, e.g., the alkyl moiety of the arylalkyl group is (C₁-C₈) alkyl and the aryl moiety is (C₆-C₁₂) aryl. In still other aspects, an arylalkyl group is (C₇-C₁₅) arylalkyl, e.g., the alkyl moiety of the arylalkyl group is (C₁-C₅) alkyl and the aryl moiety is (C₆-C₁₀) aryl.

“Arylalkenyl,” by itself or as part of another substituent, refers to an acyclic alkenyl group in which one of the hydrogen atoms bonded to a carbon atom, is replaced with an aryl group as, as defined herein. In some aspects, an arylalkenyl group is (C₈-C₄₀) arylalkenyl, e.g., the alkenyl moiety of the arylalkenyl group is (C₂-C₁₀) alkenyl and the aryl moiety is (C₆-C₃₀) aryl. In other aspects, an arylalkenyl group is (C₈-C₃₀) arylalkenyl, e.g., the alkenyl moiety of the arylalkenyl group is (C₂-C₁₀) alkenyl and the aryl moiety is (C₈-C₂₀) aryl. In other aspects, an arylalkenyl group is (C₈-C₂₀) arylalkenyl, e.g., the alkenyl moiety of the arylalkenyl group is (C₂-C₈) alkenyl and the aryl moiety is (C₆-C₁₂) aryl. In still other aspects, an arylalkenyl group is (C₈-C₁₅) arylalkenyl, e.g., the alkenyl moiety of the arylalkenyl group is (C₂-C₅) alkenyl and the aryl moiety is (C₆-C₁₀) aryl.

“Arylalkynyl,” by itself or as part of another substituent, refers to an acyclic alkynyl group in which one of the hydrogen atoms bonded to a carbon atom, is replaced with an aryl group as, as defined herein. In some aspects, an arylalkynyl group is (C₈-C₄₀) arylalkynyl, e.g., the alkynyl moiety of the arylalkynyl group is (C₂-C₁₀) alkynyl and the aryl moiety is (C₆-C₃₀) aryl. In other aspects, an arylalkynyl group is (C₈-C₃₀) arylalkynyl, e.g., the alkynyl moiety of the arylalkynyl group is (C₂-C₁₀) alkynyl and the aryl moiety is (C₆-C₂₀) aryl. In other aspects, an arylalkynyl group is (C₈-C₂₀) arylalkynyl, e.g., the alkynyl moiety of the arylalkenyl group is (C₂-C₈) alkynyl and the aryl moiety is (C₆-C₁₂) aryl. In still other aspects, an arylalkynyl group is (C₈-C₁₅) arylalkynyl, e.g., the alkynyl moiety of the arylalkynyl group is (C₂-C₅) alkynyl and the aryl moiety is (C₆-C₁₀) aryl.

“Aryldiyl,” by itself or as part of another substituent, refers to a divalent aromatic hydrocarbon group derived by the removal of two hydrogen atoms from two different carbon atoms of a parent aromatic ring system, as defined herein. Typical aryldiyl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like. In some aspects, an aryldiyl group comprises from 6 to 30 carbon atoms (C₆-C₃₀ aryldiyl). In some aspects, an aryldiyl group comprises from 6 to 20 carbon atoms (C₆-C₂₀ aryldiyl). In other aspects, an aryldiyl group comprises from 6 to 15 carbon atoms (C₆-C₁₅ aryldiyl). In still other aspects, an aryldiyl group comprises from 6 to 10 carbon atoms (C₆-C₁₀ aryldiyl).

“Arylalkyldiyl,” by itself or as part of another substituent, refers to an acyclic alkyl group in which one of the hydrogen atoms bonded to a carbon atom, typically a terminal or sp³ carbon atom, is replaced with an aryldiyl group as, as defined herein. In some aspects, an arylalkyldiyl group is (C₇-C₄₀) arylalkyldiyl, e.g., the alkyl moiety of the arylalkyldiyl group is (C₁-C₁₀) alkyl and the aryl moiety is (C₆-C₃₀) aryldiyl. In other aspects, an arylalkyldiyl group is (C₇-C₃₀) arylalkyldiyl, e.g., the alkyl moiety of the arylalkyldiyl group is (C₁-C₁₀) alkyl and the aryl moiety is (C₆-C₂₀) aryldiyl. In other aspects, an arylalkyldiyl group is (C₇-C₂₀) arylalkyldiyl, e.g., the alkyl moiety of the arylalkyldiyl group is (C₁-C₈) alkyl and the aryl moiety is (C₆-C₁₂) aryldiyl. In still other aspects, an arylalkyldiyl group is (C₇-C₁₅) arylalkyldiyl, e.g., the alkyl moiety of the arylalkyldiyl group is (C₁-C₅) alkyl and the aryl moiety is (C₆-C₁₀) aryldiyl.

“Arylalkenyldiyl,” by itself or as part of another substituent, refers to an acyclic alkyl group in which one of the hydrogen atoms bonded to a carbon atom, typically a terminal or sp² carbon atom, is replaced with an aryldiyl group as, as defined herein. In some aspects, an arylalkenyldiyl group is (C₇-C₄₀) arylalkenyldiyl, e.g., the alkenyl moiety of the arylalkenyldiyl group is (C₁-C₁₀) alkenyl and the aryl moiety is (C₆-C₃₀) aryldiyl. In other aspects, an arylalkenyldiyl group is (C₇-C₃₀) arylalkenyldiyl, e.g., the alkenyl moiety of the arylalkenyldiyl group is (C₁-C₁₀) alkenyl and the aryl moiety is (C₆-C₂₀) aryldiyl. In other aspects, an arylalkyldiyl group is (C₇-C₂₀) arylalkyldiyl, e.g., the alkenyl moiety of the arylalkenyldiyl group is (C₂-C₈) alkenyl and the aryl moiety is (C₆-C₁₂) aryldiyl. In still other aspects, an arylalkyldiyl group is (C₇-C₁₅) arylalkyldiyl, e.g., the alkenyl moiety of the arylalkenyldiyl group is (C₁-C₅) alkenyl and the aryl moiety is (C₆-C₁₀) aryldiyl.

“Compounds,” refers to compounds encompassed by structural formulae disclosed herein and includes any specific compounds within these formulae whose structure is disclosed herein. Compounds may be identified either by their chemical structure and/or chemical name. The chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass the stereoisomerically pure form depicted in the structure (e.g., geometrically pure, enantiomerically pure or diastereomerically pure). The chemical structures depicted herein also encompass the enantiomeric and stereoisomeric derivatives of the compound depicted. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds may also be atropisomers. The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Cycloalkyl,” by itself or as part of another substituent, refers to a saturated cyclic monovalent hydrocarbon radical derived by the removal of one hydrogen atom from a single carbon atom of a parent cycloalkane. Typical cycloalkyl groups include, but are not limited to, cyclopropyl, cyclobutyl, cyclopentyl; etc.; and the like. In some aspects, a cycloalkyl group comprises from 3 to 20 carbon atoms (C₃-C₁₅ cycloalkyl). In other aspects, a cycloalkyl group comprises from 3 to 10 carbon atoms (C₃-C₁₀ cycloalkyl). In still other aspects, a cycloalkyl group comprises from 3 to 8 carbon atoms (C₃-C₈ cycloalkyl). The term “cyclic monovalent hydrocarbon radical” also includes multicyclic hydrocarbon ring systems having a single radical and between 5 and 12 carbon atoms. Exemplary multicyclic cycloalkyl rings include, for example, norbornyl, pinyl, and adamantyl.

“Cycloalkenyl,” by itself or as part of another substituent, refers to an unsaturated cyclic monovalent hydrocarbon radical derived by the removal of one hydrogen atom from a single carbon atom of a parent cycloalkene. Typical cycloalkenyl groups include, but are not limited to, cyclopropenyl, cyclobutenyl, cyclopentenyl; etc.; and the like. In some aspects, a cycloalkenyl group comprises from 3 to 20 carbon atoms (C₃-C₂₀ cycloalkenyl). In other aspects, a cycloalkenyl group comprises from 3 to 10 carbon atoms (C₃-C₁₀ cycloalkenyl). In still other aspects, a cycloalkenyl group comprises from 3 to 8 carbon atoms (C₃-C₈ cycloalkenyl).

“Cycloalkyldiyl,” by itself or as part of another substituent, refers to a saturated cyclic divalent hydrocarbon radical derived by the removal of two hydrogen atom from one or two carbon atoms of a parent cycloalkane. Typical cycloalkyldiyl groups include, but are not limited to, cyclopropyldiyl, cyclobutyldiyl, cyclopentyldiyl; etc.; and the like. In some aspects, a cycloalkyldiyl group comprises from 3 to 20 carbon atoms (C₃-C₁₅ cycloalkyldiyl). In other aspects, a cycloalkyldiyl group comprises from 3 to 10 carbon atoms (C₃-C₁₀ cycloalkyldiyl). In still other aspects, a cycloalkyldiyl group comprises from 3 to 8 carbon atoms (C₃-C₈ cycloalkyldiyl). The term “cyclic divalent hydrocarbon radical” also includes multicyclic hydrocarbon ring systems having two radicals and between 5 and 12 carbon atoms. Exemplary multicyclic cycloalkyl rings include, for example, norbornyl, pinyl, and adamantyl.

“Cycloheteroalkyl,” by itself or as part of another substituent, refers to a cycloalkyl group as defined herein in which one or more of the carbon atoms (and optionally any associated hydrogen atoms), are each, independently of one another, replaced with the same or different heteroatoms or heteroatomic groups as defined in “heteroalkyl” below. In some aspects, a cycloheteroalkyl group comprises from 3 to 20 carbon and hetero atoms (3-20 cycloheteroalkyl). In other aspects, a cycloheteroalkyl group comprises from 3 to 10 carbon and hetero atoms (₃₋₁₀ cycloheteroalkyl). In still other aspects, a cycloheteroalkyl group comprises from 3 to 8 carbon and hetero atoms (₃₋₈ cycloheteroalkyl). The term “cyclic monovalent heteroalkyl radical” also includes multicyclic heteroalkyl ring systems having a single radical and between 3 and 12 carbon and at least one hetero atom. Exemplary cycloheteroalkyl groups include, for example, azetidine, pyrrolidine, piperazine, piperidine, morpholine and tetrahydrofuran.

“Cycloheteroalkyldiyl,” by itself or as part of another substituent, refers to a cycloalkyldiyl group as defined herein in which one or more of the carbon atoms (and optionally any associated hydrogen atoms), are each, independently of one another, replaced with the same or different heteroatoms or heteroatomic groups as defined in “heteroalkyl” below. In some aspects, a cycloheteroalkyldiyl group comprises from 3 to 20 carbon and hetero atoms (₃₋₂₀ cycloheteroalkyldiyl). In other aspects, a cycloheteroalkyldiyl group comprises from 3 to 10 carbon and hetero atoms (₃₋₁₀ cycloheteroalkyldiyl). In still other aspects, a cycloheteroalkyldiyl group comprises from 3 to 8 carbon and hetero atoms (3-8 cycloheteroalkyldiyl). The term “cyclic divalent heteroalkyl radical” also includes multicyclic heteroalkyl ring systems having two radicals and between 3 and 12 carbon and at least one hetero atom. Exemplary cycloheteroalkyl groups include, for example, azetidine, pyrrolidine, piperazine, piperidine, morpholine and tetrahydrofuran.

“Cycloheteroalkenyl,” by itself or as part of another substituent, refers to a cycloalkenyl group as defined herein in which one or more of the carbon atoms (and optionally any associated hydrogen atoms), are each, independently of one another, replaced with the same or different heteroatoms or heteroatomic groups as defined in “heteroalkenyl” below. In some aspects, a cycloheteroalkenyl group comprises from 3 to 20 carbon and hetero atoms (₃₋₂₀ cycloheteroalkenyl). In other aspects, a cycloheteroalkenyl group comprises from 3 to 10 carbon and hetero atoms (₃₋₁₀) cycloheteroalkenyl). In still other aspects, a cycloheteroalkenyl group comprises from 3 to 8 carbon and heteroatoms (3-8 cycloheteroalkenyl). The term “cyclic monovalent heteroalkenyl radical” also includes multicyclic heteroalkenyl ring systems having a single radical and between 2 and 12 carbon and at least one hetero atom.

“Halo,” by itself or as part of another substituent refers to a radical —F, —Cl, —Br or —I.

“Heteroalkyl,” refer to an alkyl group, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷ R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently some aspects, an heteroalkyl group comprises from 1 to 20 carbon and hetero atoms (1-20 heteroalkyl). In other aspects, an heteroalkyl group comprises from 1 to 10 carbon and hetero atoms (₁₋₁₀ heteroalkyl). In still other aspects, an heteroalkyl group comprises from 1 to 6 carbon and hetero atoms (₁₋₆ heteroalkyl).

Heteroalkyldiyl” by itself or as part of another substituent, refers to a saturated, branched or straight-chain heteroalkyl group derived by the removal of one hydrogen atom from each of two different carbon atoms of a parent heteroalkane, or by the removal of two hydrogen atoms from a single carbon atom of a parent heteroalkane. The two monovalent radical centers or each valency of the divalent radical center can form bonds with the same or different atoms. In some embodiments, the heteroalkyldiyl group is (₁₋₂₀) heteroalkyldiyl. In other embodiments, the heteroalkyldiyl group is (₁₋₁₀) heteroalkyldiyl. In still other embodiments, the heteroalkyldiyl group is (₁₋₆) heteroalkyldiyl.

Heteroalkyltriyl” by itself or as part of another substituent, refers to a saturated, branched or straight-chain heteroalkyl group derived by the removal of three hydrogen atom from carbon atom of a parent heteroalkane. The three monovalent radical centers can form bonds with the same or different atoms. In some embodiments, the heteroalkyltriyl group is (C₁-C₂₀) heteroalkyltriyl. In other embodiments, the heteroalkyltriyl group is (C₁-C₁₀) heteroalkyltriyl. In still other embodiments, the heteroalkyltriyl group is (C₁-C₆) heteroalkyltriyl.

“Heteroalkenyl,” refers to a alkenyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N—N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷ R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently some aspects, an heteroalkenyl group comprises from 1 to 20 carbon and hetero atoms (₁₋₂₀ heteroalkenyl). In other aspects, an heteroalkenyl group comprises from 1 to 10 carbon and hetero atoms (₁₋₁₀ heteroalkenyl). In still other aspects, an heteroalkenyl group comprises from 1 to 6 carbon and hetero atoms (₁₋₆ heteroalkenyl).

“Heteroalkenyldiyl” by itself or as part of another substituent, refers to a unsaturated, branched or straight-chain heteroalkenyl group derived by the removal of one hydrogen atom from each of two different carbon atoms of a parent heteroalkene, or by the removal of two hydrogen atoms from a single carbon atom of a parent heteroalkene. The two monovalent radical centers or each valency of the divalent radical center can form bonds with the same or different atoms. In some embodiments, the heteroalkenyldiyl group is (C₂-C₂₀) heteroalkenyldiyl. In other embodiments, the heteroalkenyldiyl group is (C₂-C₁₀) heteroalkenyldiyl. In still other embodiments, the alkenyldiyl group is (C₂-C₆) alkenyldiyl.

“Heteroaryl,” by itself or as part of another substituent, refers to a monovalent heteroaromatic radical derived by the removal of one hydrogen atom from a single atom of a parent heteroaromatic ring system, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryl group comprises from 5 to 20 ring atoms (5-20 membered heteroaryl). In other aspects, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. In some aspects, the heteroarylalkyl group is a 6-21 membered heteroarylalkyl, e.g., the alkyl moiety of the heteroarylalkyl is (C₁-C₆) alkyl and the heteroaryl moiety is a 5-15-membered heteroaryl. In other aspects, the heteroarylalkyl is a 6-13 membered heteroarylalkyl, e.g., the alkyl moiety is (C₁-C₃) alkyl and the heteroaryl moiety is a 5-10 membered heteroaryl.

“Heteroarylalkenyl,” by itself or as part of another substituent refers to an acyclic alkenyl group in which one of the hydrogen atoms bonded to a carbon atom, is replaced with a heteroaryl group. In some aspects, the heteroarylalkenyl group is a 7-21 membered heteroarylalkenyl, e.g., the alkenyl moiety of the heteroarylalkenyl is (C₂-C₆) alkenyl and the heteroaryl moiety is a 5-15-membered heteroaryl. In other aspects, the heteroarylalkenyl is a 7-13 membered heteroarylalkenyl, e.g., the alkenyl moiety is (C₂-C₃) alkenyl and the heteroaryl moiety is a 5-10 membered heteroaryl.

“Heteroarylalkynyl,” by itself or as part of another substituent refers to an acyclic alkenyl group in which one of the hydrogen atoms bonded to a carbon atom, is replaced with a heteroaryl group. In some aspects, the heteroarylalkynyl group is a 7-21 membered heteroarylalkynyl, e.g., the alkynyl moiety of the heteroarylalkynyl is (C₂-C₆) alkynyl and the heteroaryl moiety is a 5-15-membered heteroaryl. In other aspects, the heteroarylalkynyl is a 7-13 membered heteroarylalkynyl, e.g., the alkynyl moiety is (C₂-C₃) alkynyl and the heteroaryl moiety is a 5-10 membered heteroaryl.

“Heteroaryldiyl,” by itself or as part of another substituent, refers to a divalent radical derived by the removal of two hydrogen atoms from two different atoms of a parent heteroaromatic ring system, as defined herein. Typical heteroaryldiyl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some aspects, the heteroaryldiyl group comprises from 5 to 20 ring atoms (5-20 membered heteroaryldiyl). In other aspects, the heteroaryldiyl group comprises from 5 to 10 ring atoms (5-10 membered heteroaryldiyl). Exemplary heteroaryldiyl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyldiyl,” by itself or as part of another substituent refers to an acyclic alkyl group in which one of the hydrogen atoms bonded to a carbon atom, typically a terminal or sp³ carbon atom, is replaced with a heteroaryldiyl group. In some aspects, the heteroarylalkyldiyl group is a 6-21 membered heteroarylalkyldiyl, e.g., the alkyl moiety of the heteroarylalkyldiyl is (C₁-C₆) alkyl and the heteroaryldiyl moiety is a 5-15-membered heteroaryl. In other aspects, the heteroarylalkyldiyl is a 6-13 membered heteroarylalkyldiyl, e.g., the alkyl moiety is (C₁-C₃) alkyl and the heteroaryldiyl moiety is a 5-10 membered heteroaryldiyl.

Heteroarylalkenyldiyl,” by itself or as part of another substituent refers to an acyclic alkenyl group in which one of the hydrogen atoms bonded to a carbon atom, is replaced with a heteroaryldiyl group. In some aspects, the heteroarylalkenyldiyl group is a 7-21 membered heteroarylalkenyldiyl, e.g., the alkenyl moiety of the heteroarylalkenyl is (C₂-C₆) alkenyl and the heteroaryl moiety is a 5-15-membered heteroaryldiyl. In other aspects, the heteroarylalkenyl is a 7-13 membered heteroarylalkenyldiyl, e.g., the alkenyl moiety is (C₂-C₃) alkenyl and the heteroaryldiyl moiety is a 5-10 membered heteroaryldiyl.

“Hydrates,” refers to incorporation of water into to the form of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See e.g., (Guillory, K., Chapter 5, pp. 202-205 in Polymorphism in Pharmaceutical Solids, (Brittain, H. ed.), Marcel Dekker, Inc., New York, NY, 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of hydrates such as, for example, HOLODIAG, Pharmaparc II, Voie de l'Innovation, 27 100 Val de Reuil, France (http://www.holodiag.com).

“N-oxide,” refers to a compound containing an N—O bond with three additional hydrogen or side chains attached to the N, or a compound containing an N—O bond with two additional hydrogen or side chains attached to the N, so that there is a positive charge on the nitrogen. The N-oxides of the present disclosure can be synthesized by oxidation procedures well known to those skilled in the art.

“Parent Aromatic Ring System,” refers to an unsaturated cyclic or polycyclic ring system having a conjugated pi electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System,” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, b-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Salt,” refers to a salt of acrosslinker. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Protecting group,” refers to a grouping of atoms that when attached to a reactive functional group in a molecule masks, reduces or prevents reactivity of the functional group during chemical synthesis. Examples of protecting groups can be found in Green et al., “Protective Groups in Organic Chemistry,” (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Solvates,” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See e.g., (Guillory, K., Chapter 5, pp. 202-205 in Polymorphism in Pharmaceutical Solids, (Brittain, H. ed.), Marcel Dekker, Inc., New York, NY, 1999)). The above methods for preparing solvates are well within the ambit of those of skill in the art, are completely conventional and do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of solvates such as, for example, HOLODIAG, Pharmaparc II, Voie de l'Innovation, 27 100 Val de Reuil, France (http://www.holodiag.com).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include R^{a}, halo, —O⁻, —O, —OR^{b}, —SR^{b}, —S—, ═S, —NR^{c}R^{c}, —NR^{b}, —N—OR^{b}, trihalomethyl, —CF₃, —CN, —OCN, —SCN, —NO, —NO₂, —N—OR^{b}, —N—NR^{c}R^{c}, —NR^{b}S(O)₂R^{b}, ═N₂, —N₃, —S(O)₂R^{b}, —S(O)₂NR^{b}R^{b}, —S(O)₂O⁻, —S(O)₂OR^{b}, —OS(O)₂R^{b}, —OS(O)₂O⁻, —OS(O)₂OR^{b}, —OS(O)₂NR^{c}NR^{c}, —P(O)(O⁻)₂, —P(O)(OR^{b})(O⁻), —P(O)(OR^{b})(OR^{b}), —C(O)R^{b}, —C(O)NR^{b}—OR^{b}—C(S) R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(O)NR^{c}R^{c}, —OC(NCN)NR^{c}R^{c}OC(S)OR^{b}, —NR^{b}C(O)R^{b}, —NR^{b}C(S)R^{b}, —NR^{b}C(O)O⁻, —NR^{b}C(O)OR^{b}, —NR^{b}C(NCN)OR^{b}, —NR^{b}S(O)₂NR^{c}R^{c}, —NR^{b}C(S)O R^{b}, —NR^{b}C(O)NR^{c}R^{c}, —NR^{b}C(S)NR^{c}R^{c}, —NR^{b}C(S)NR^{b}C(O)R^{a}, —NR^{b}S(O)₂OR^{b}, —NR^{b}S(O)₂R^{b}, —NR^{b}C(NCN)NR^{c}R^{c}, —NR^{b}C(NR^{b})R^{b} and —NR^{b}C(NR^{b})NR^{c}R^{c}, where each R^{a} is independently, substituted alkyl, substituted alkenyl, substituted alkynyl, aryl, substituted aryl, heteroalkyl, substituted heteroalkyl, heteroalkenyl, substituted heteroalkenyl, heteroalkynyl, substituted heteroalkynyl, heteroaryl or substituted heteroaryl; each R^{b} is independently hydrogen, substituted alkyl, substituted alkenyl, substituted alkynyl, heteroalkyl, substituted heteroalkyl, heteroalkenyl, substituted heteroalkenyl, heteroalkynyl, substituted heteroalkynyl, arylalkyl, substituted arylalkyl, arylalkenyl, substituted arylalkenyl, arylalkynyl, substituted arylalkynyl, heteroarylalkyl, substituted heteroarylalkyl, heteroarylalkenyl, substituted heteroarylalkenyl, heteroarylalkynyl or substituted heteroarylalkynyl; and each R^{c} is independently R^{b} or alternatively, the two R^{c}s taken together with the nitrogen atom to which they are bonded form a 4-, 5-, 6- or 7 membered-cycloheteroalkyl, substituted cycloheteroalkyl, cycloheteroalkenyl, substituted cycloheteroalkenyl ring or a cycloheteroalkyl or cycloheteroalkenyl fused with an aryl group which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. In other aspects, substituent groups useful for substituting saturated carbon atoms in the specified group or radical include R^{a}, halo, —OR^{b}, —NR^{c}R^{c}, trihalomethyl, —CN, —NR^{b}S(O)₂R^{b}, —C(O)R^{b}, —C(O)NR^{b}—OR^{b}, —C(O)OR^{b}, —C(O)NR^{c}R^{c}, —OC(O) R^{b}, —OC(O)OR^{b}, —OS(O)₂NR^{c}NR^{c}, —OC(O)NR^{c}R^{c}, and —NR^{b}C(O)OR^{b}, where R^{a}, R^{b} and R^{c} are as previously defined. In still other aspects, substituent groups useful for substituting saturated carbon atoms in the specified group or radical include R^{a}, halo, —OR^{b}, —NR^{c}R^{c}, trihalomethyl, —CN, —C(O)R^{b}, —C(O)OR^{b}, —C(O)NR^{c}R^{c}, —OC(O)R^{b}, —OC(O)NR^{c}R^{c}, and —NR^{b}C(O)OR^{b}, where R^{a}, R^{b} and R^{c} are as previously defined.

Substituent groups useful for substituting unsaturated carbon atoms in the specified group or radical include substituted alkyl, —R^{a}, halo, —O⁻, —OR^{b}, —SR^{b}, —S—, —NR^{c}R^{c}, trihalomethyl, —CF₃, —CN, —OCN, —SCN, —NO, —NO₂, —N₃, —S(O)₂₀, —S(O)₂OR^{b}, —OS(O)₂R^{b}, —O S(O)₂OR^{b}, —OS(O)₂O, —P(O)(O)₂, —P(O)(OR^{b})(O—), —P(O)(OR^{b})(OR^{b}), —C(O)R^{b}, —C(S)R^{b}, —C(NR^{b})R^{b}, —C(O)O, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —OC(O)NR^{c}R^{c}, —OS(O)₂NR^{c}NR^{c}, —NR^{b}C(O)R^{b}, —NR^{b}C(S) R^{b}, —NR^{b}C(O)O⁻, —NR^{b}C(O)OR^{b}, —NR^{b}S(O)₂OR^{a}, —NR^{b}S(O)₂R^{a}, —NR^{b}C(S)OR^{b}, —NR^{b}C(O)NR CRC, —NR^{b}C(NR^{b})R^{b}, —NR^{b}C(NR^{b})NR^{c}R^{c} and —C(NR^{b})NR^{b}C(NR^{b})NR^{c}R^{c} where R^{a}, R^{b} and R^{c} are as previously defined. In other aspects, substituent groups useful for substituting unsaturated carbon atoms in the specified group or radical include substituted alkyl, —R^{a}, halo, —OR^{b}, —SR^{b}, —NR^{c}R^{c}, trihalomethyl, —CN, —S(O)₂OR^{b}, —C(O)R^{b}, —C(O)OR^{b}, —C(O)NR^{c}R^{c}, —OC(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting unsaturated carbon atoms in the specified group or radical include substituted alkyl, —R^{a}, halo, —OR^{b}, —NR^{c}R^{c}, trihalomethyl, —S(O)₂OR^{b}, —C(O)R^{b}, —C(O)OR^{b}, —C(O)NR^{c}R^{c}, —OC(O)R^{b}, —NR^{b}C(O)R^{b} and —NR^{b}C(O)OR^{b}, where R^{a}, R^{b} and R^{c} are as previously defined.

Substituent groups useful for substituting nitrogen atoms in heteroalkyl and cycloheteroalkyl groups include, but are not limited to, alkyl, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O, —S(O)₂OR^{b}, —OS(O)₂R^{b}, —OS(O)₂O, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S) OR^{b}, —NR^{o}C. (O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b}, —NR^{b}C(NR^{b})NR^{c}R^{c} and —C(NR^{b})NR^{b}C(NR^{b})NR^{c}R^{c} where R^{a}, R^{b} and R^{c} are as previously defined. In some aspects, substituent groups useful for substituting nitrogen atoms in heteroalkyl and cycloheteroalkyl groups include, alkyl, R^{a}, halo, —OR^{b}, —NR^{c}R^{c}, trihalomethyl, —CN, —S(O)₂OR^{b}, —OS(O)₂R^{b}, —C(O)R^{b}, —C(NR^{b})R^{b}, —C(O)OR^{b}, —C(O)NR^{c}R^{c}, —O C(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined. In still other aspects, substituent groups useful for substituting nitrogen atoms in heteroalkyl and cycloheteroalkyl groups include, alkyl, R^{a}, halo, —OR^{b}, —NR^{c}R^{c}, trihalomethyl, —CN, —S(O)₂OR^{b}, —C(O)R^{b}, —C(NR^{b})R^{b}, —C(O)OR^{b}, —C(O)NR^{c}R^{c}, —OC(O)R^{b}, —NR^{b}C(O)R^{b} and —NR^{o}C(O)OR^{b}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

### Crosslinkers

In one aspect, a compound of formula (I):

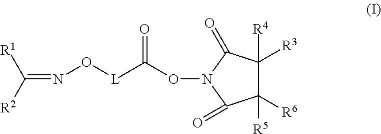

or salts, hydrate or solvates thereof wherein: R¹ and R² are independently alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, heterocycloalkyl, substituted heterocycloalkyl, heteroalkyl, substituted heteroalkyl, heteroalkenyl, substituted heteroalkenyl, heteroaryl, substituted heteroaryl, heteroarylalkyl, substituted heteroarylalkyl, heteroarylalkenyl, substituted heteroarylalkenyl, or together with the atoms to which they are bonded form a cycloalkyl, substituted cycloalkyl, cycloalkenyl, substituted cycloalkenyl, heterocycloalkyl, substituted heterocycloalkyl, heterocycloalkenyl or substituted heterocycloalkenyl ring; R³, R⁴, R⁵ and R⁶ are independently —H, alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, heterocycloalkyl, substituted heterocycloalkyl, heteroalkyl, substituted heteroalkyl, heteroalkenyl, substituted heteroalkenyl, heteroaryl, substituted heteroaryl, heteroarylalkyl, substituted heteroarylalkyl, heteroarylalkenyl, substituted heteroarylalkenyl or optionally one of R³, R⁴, R⁵ and R⁶ is —SO₃H or a salt thereof; L is alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl, substituted heteroarylalkenyldiyl, a complementary oligonucleotide between 1 and 30 nucleic acid bases, a complementary oligopeptide between 1 and 30 amino acids or -L₁X(L₂Z)ₐL₃-; L₁, L₂ and L₃ are independently alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl or substituted heteroarylalkenyldiyl; X is alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl, substituted heteroarylalkenyldiyl, alkyltriyl, heteroalkyltriyl, substituted heteroalkyltriyl, a complementary oligopeptide between 1 and 30 amino acids, a complementary oligonucleotide between 1 and 30 nucleic acid bases, —(CH₂)S—S(CH₂)ᵣ—, —(CH₂)ᵣ(OCH₂CH₂)ₛ(CH₂)ᵣ—, —(CH₂)ᵣC(O)(CH₂)ᵣ—, —(CH₂)ᵣC(O)₂(CH₂)ᵣ—, —(CH₂)ᵣS(CH₂)ᵣ— or —(CH₂)ᵣSO₂(CH₂)ᵣ—; each r is independently an integer between 3 and 25; Z is maleimide, Texas Red, Alexa Fluor 48, Fluorescein isothiocyanate, Pyridine disulfide, Rhodamine, Rhodamine B, Tetramethyl Rhodamine, N₃, alkyne,

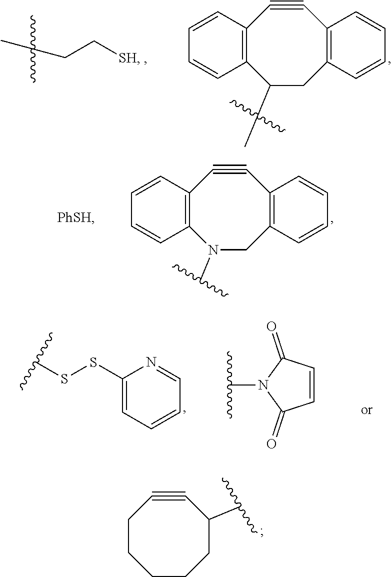

and a is 0 or 1 is provided.

The compounds of Formula (I) can be made by methods known to those of skill in the art, some of which are exemplified in the examples provided below.

In some embodiments, R¹ and R² are independently alkyl, alkenyl, aryl, or arylalkyl. In other embodiments, R¹ and R² are independently —CH₃, —C₂H₅, —C₃H₇, cyclopentyl, cyclohexyl, allyl, phenyl or benzyl. In still other embodiments, R¹ and R² are independently —CH₃, —C₂H₅, or phenyl.

In some embodiments, R³, R⁴, R⁵ and R⁶ are independently —H, alkyl, alkenyl, aryl, arylalkyl or —SO₃H or a salt thereof. In other embodiments, R³, R⁴, R⁵ and R⁶ are independently —H, —CH₃, —C₂H₅, —C₃H₇, allyl, phenyl, benzyl or —SO₃H or a salt thereof. In still other embodiments, R³, R⁴, R⁵ and R⁶ are independently —H, —CH₃ or —SO₃H or a salt thereof. In still other embodiments, R³, R⁴, R⁵ and R⁶ are independently —H, or —SO₃H or a salt thereof.

In some embodiments, L is —(CH₂)ₙ—, —(CH₂CH₂O)ₙ—, —(CH(CH₃)CH₂O)ₙ, —(CH(CH₃)CH₂O)ₙCH₂—, —(CH₂)ₙO—, —C(O)(C H₂)ₙO—, —C(O)(CH₂)ₙNH—, —C(O)(CHR₇)(NHC(O)(CHR₇)ₙNH—, —CH₂C(O)(CH₂)ₙO—, —CH₂C(O)(CH₂)ₙNH—, —CH₂C(O)(CHR₇) NHC(O)CHR₇)ₙNH—, —(CH₂)₃(CH₂)ₙNR⁸CH₂—, —(CH₂)₃(CH₂)ₙNR⁸(CH₂)₃—, —(CH₂)₃(CH₂)ₙNR⁸—, or —(CH₂)₃(CH₂)ₙNR⁸C(O)CH₂—; a complementary oligonucleotide between 1 and 30 nucleic acid bases; a complementary oligopeptide between 1 and 30 amino acids; each R⁷ is independently —H, (C₁-C₅) alkyl, (C₁-C₅) substituted alkyl, aryl, substituted aryl, arylalkyl, substituted arylalkyl, heteroarylalkyl or heteroaryl; R⁸ is —H or alkyl; and n is an integer between 1 and 30. In other embodiments, a is 0, L is L₁-X-L₃ and L₁ and L₃ are independently —(CH₂)ₒC(O)OXOC(O)(CH₂)ₒ—, —(CH₂)ₒC(O)NHXNHC(O)(CH₂)ₒ—, —(CH₂)ₒX(CH₂)ₒ—, —(CH₂)ₒX(CH₂)ₒO—, —(CH₂)ₒX(CH₂)ₒNH—, —C(O)(CH₂)ₒX(CH₂)ₒO—, —C(O)(CH₂)ₒX(CH₂)ₒNH—, —(CH₂)₂C(O)NHXNHC(O)(CH₂)₂—, —(CH₂)₂C(O)NHXC(O)NH(CH₂)₂—, —(CH₂)₂C(O)NHXOC(O)(CH₂)₂—, —(CH₂)₂C(O)NHX(O)O(CH₂)₂—, or —(CH₂)₂C(O)OXOC(O)(CH₂)₂—; X is alkyldiyl, substituted alkyldiyl, alkenyldiyl, substituted alkenyldiyl, aryldiyl, substituted aryldiyl, cycloalkyldiyl, substituted cycloheteroalkyldiyl, cycloheteroalkyldiyl, substituted cycloalkyldiyl, alkenyldiyl, substituted alkenyldiyl, heteroalkyldiyl, substituted heteroalkyldiyl, heteroalkenyldiyl, substituted heteroalkenyldiyl, heteroaryldiyl, substituted heteroaryldiyl; and o is an integer between 1 and 15. In still other embodiments, a is 1, L is L₁-X(L₂Z)-L₃; L₁, L₂ and L₃ are independently —(CH₂)ₙ—, —(CH₂CH₂O)ₙ—, —(CH₂))ₒ—C(O)OXOC(O)(CH₂)ₒ—, —(CH₂))ₒ—C(O)NHXNHC(O)(CH₂)ₒ—, —(CH₂))ₒ—X(CH₂)ₒ—, —(CH₂))ₒ—X(CH₂))ₒ—O—, —(CH₂))ₒ—X(CH₂)ₒNH—, —C(O)(CH₂))ₒ—X(CH₂))ₒ—O—, —C(O)(CH₂))ₒ—X(CH₂))ₒ—NH—, —(CH₂)₂C(O)NHXNHC(O)(CH₂)₂—, —(CH₂)₂C(O)NHXC(O)NH(CH₂)₂—, —(CH₂)₂C(O)NHXOC(O)(CH₂)₂—, —(CH₂)₂C(O)NHX(O)O(CH₂)₂—, —(CH₂)₂C(O)OXOC(O)(CH₂)₂—; a complementary oligopeptide between 1 and 30 amino acids; a complementary oligonucleotide between 1 and 30 nucleic acid bases; X is alkyltriyl, heteroalkyltriyl, substituted heteroalkyltriyl; and o is an integer between 1 and 15. In still other embodiments, L₁, L₂, and L₃ are independently C₁-C₂₀ alkyl, phenyl, aryl, —NH—, —C(O)—, —(CH₂)ₙ—NH—C(O)—, —(CH₂)ₙ—C(O)—NH—, —(CH₂—CH₂—O)ₙ—, —(O—CH₂—CH(CH₃))ₙ—C(O)—NH—, or —(OCH₂—CH(CH₃))ₙ—; and each n is independently an integer from 1 to 25.

In still other embodiments, R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof and L is —(CH₂)ₙ—, —(CH₂CH₂O)ₙ—, —(CH(CH₃)CH₂O)ₙ, —(CH(CH₃)CH₂O)ₙCH₂—, —(CH₂)ₙO—, —C(O)(C H₂)ₙO—, —C(O)(CH₂)ₙNH—, —C(O)(CHR₇) (NHC(O)(CHR₇)ₙNH—, —CH₂C(O)(CH₂)ₙO—, —CH₂C(O)(CH₂)ₙNH—, —CH₂C(O)(CHR₇) NHC(O)CHR₇)ₙNH—, —(CH₂)₃(CH₂)ₙNR⁸CH₂—, —(CH₂)₃(CH₂)ₙNR⁸ (CH₂)₃—, —(CH₂)₃(CH₂)ₙNR⁸—, —(CH₂)₃(CH₂)ₙNR⁸C(O)CH₂—, a complementary oligonucleotide between 1 and 30 nucleic acid bases, a complementary oligopeptide between 1 and 30 amino acids; each R⁷ is independently —H, (C₁-C₅) alkyl, (C₁-C₅) substituted alkyl, aryl, substituted aryl, arylalkyl, substituted arylalkyl, heteroarylalkyl or heteroaryl; R⁸ is —H or alkyl; and n is an integer between 1 and 30. In other embodiments, R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

In some embodiments, R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof, a is 0, L is L₁-X-L₃, L₁ and L₃ are independently —(CH₂)ₒC(O)OXOC(O)(CH₂)ₒ—, —(CH₂)ₒC(O)NHXNHC(O)(CH₂)ₒ—, —(CH₂)ₒX(CH₂)ₒ—, —(CH₂)ₒX(CH₂)ₒO—, —(CH₂)ₒX(CH₂)ₙNH—, —C(O)(CH₂)ₒX(CH₂)ₒO—, —C(O)(CH₂)ₒX(CH₂)ₒNH—, —(CH₂)₂C(O)NHXNHC(O)(CH₂)₂—, —(CH₂)₂C(O)NHXC(O)NH(CH₂)₂—, —(CH₂)₂C(O)NHXOC(O)(CH₂)₂—, —(CH₂)₂C(O)NHX(O)O(CH₂)₂—, or —(CH₂)₂C(O)OXOC(O)(CH₂)₂—; X is alkyldiyl, substituted alkyldiyl, alkenyldiyl, substituted alkenyldiyl, aryldiyl, substituted aryldiyl, cycloalkyldiyl, substituted cycloheteroalkyldiyl, cycloheteroalkyldiyl, substituted cycloalkyldiyl, alkenyldiyl, substituted alkenyldiyl, heteroalkyldiyl, substituted heteroalkyldiyl, heteroalkenyldiyl, substituted heteroalkenyldiyl, heteroaryldiyl, substituted heteroaryldiyl; and o is an integer between 1 and 15. In other embodiments, R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

In some embodiments, R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof, a is 1, L is L₁-X(L₂Z)-L₃ and L₁, L₂ and L₃ are independently —(CH₂)ₙ—, —(CH₂CH₂O)ₙ—, —(CH₂)ₒC(O)OXOC(O)(CH₂)ₒ—, —(CH₂)ₒC(O)NHXNHC(O)(CH₂)ₒ—, —(CH₂)ₒX(CH₂)ₒ—, —(CH₂)ₒX(CH₂)ₒO—, —(CH₂) X(CH₂)ₒNH—, —C(O)(CH₂)ₒX(CH₂)ₒO—, —C(O)(CH₂)ₒX(CH₂)ₒNH—, —(CH₂)₂C(O)NHXNHC(O)(CH₂)₂—, —(CH₂)₂C(O)NHXC(O)NH(CH₂)₂—, —(CH₂)₂C(O)NHXOC(O)(CH₂)₂—, —(CH₂)₂C(O)NHX(O)O(CH₂)₂—, -or (CH₂)₂C(O)OXOC(O)(CH₂)₂—; a complementary oligopeptide between 1 and 30 amino acids; a complementary oligonucleotide between 1 and 30 nucleic acid bases, X is alkyltriyl, heteroalkyltriyl, substituted heteroalkyltriyl; and o is an integer between 1 and 15. In other embodiments, R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

In some embodiments, R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof, L₁, L₂, and L₃ are independently C₁-C₂₀ alkyl, phenyl, aryl, —NH—, —C(O)—, —(CH₂)ₙ—NH—C(O)—, —(CH₂)ₙ—C(O)—NH—, —(CH₂—CH₂—O)ₙ—, —(O—CH₂—CH(CH₃))ₙ—C(O)—NH—, or —(OCH₂—CH(CH₃))ₙ—; and each n is independently an integer from 1 to 25. In other embodiments, R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

In some embodiments, L₁, L₂ and L₃ are independently

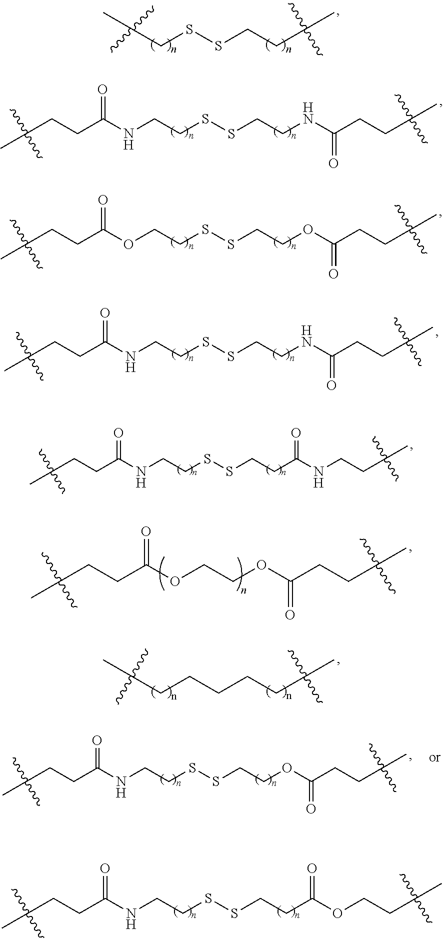

wherein n is an integer from 1 to 25.

The compound any one of claims 1-8, wherein L₁, L₂ and L₃ are independently

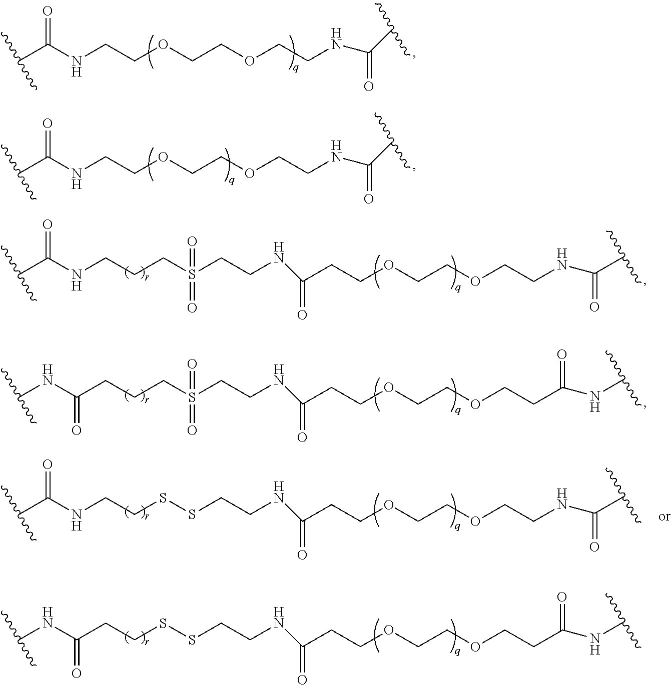

wherein each q and r are independently an integer between 1 and 25.

The compound of any one of claims 1-8, wherein X is

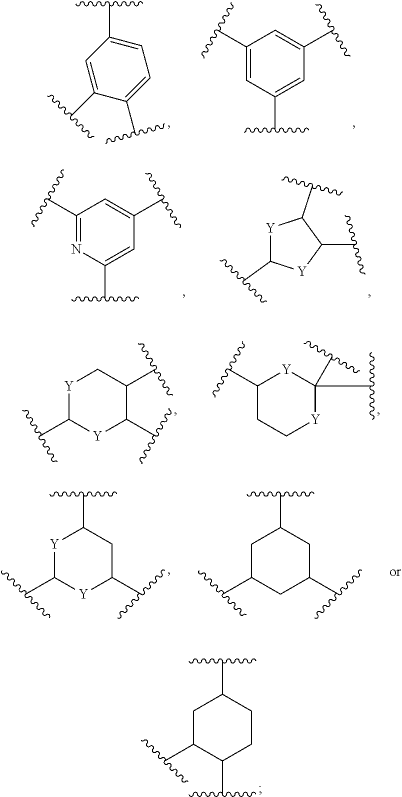

and each Y is independently —O—, —S—, —CH₂— or NR⁷; and R⁷ is —H or alkyl.

In some embodiments, X is

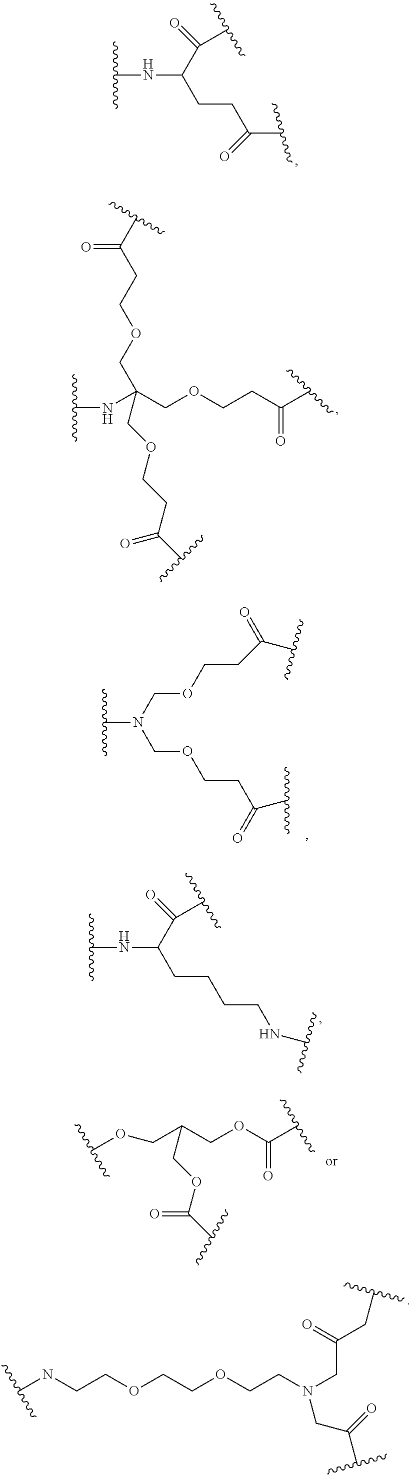 [SMILES: *NC(CCC(*)=O)C(*)=O ; *NC(COCCC(*)=O)(COCCC(*)=O)COCCC(*)=O ; *C(=O)CCOCN(*)COCCC(*)=O ; *NCCCCC(N*)C(*)=O ; *OCC(COC(*)=O)COC(*)=O ; *CC(=O)CN(CCOCCOCCN*)CC(*)=O]

In some embodiments, a compound of structure:

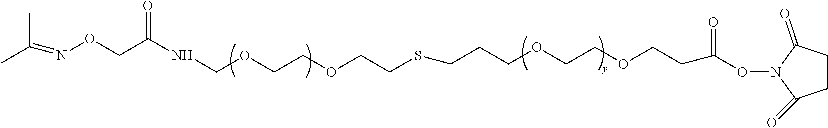

wherein x and y are independently integers from 1 to 25 is provided.

In some embodiments, a compound of structure:

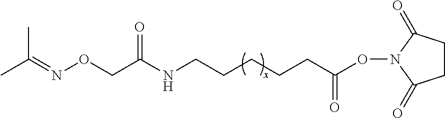

wherein x is an integer from 1-25 is provided.

In some embodiments, a compound of structure:

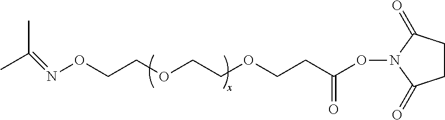

wherein x is an integer from 1-25 is provided.

In some embodiments, a compound of structure:

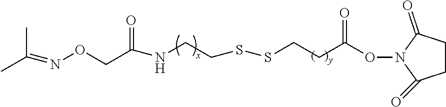

wherein x is an integer from 1-25 is provided.

In some embodiments, a compound of structure:

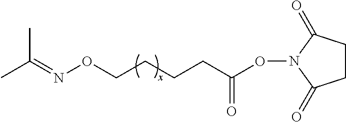

wherein x is an integer from 1-25 is provided.

In some embodiments, a compound of structure:

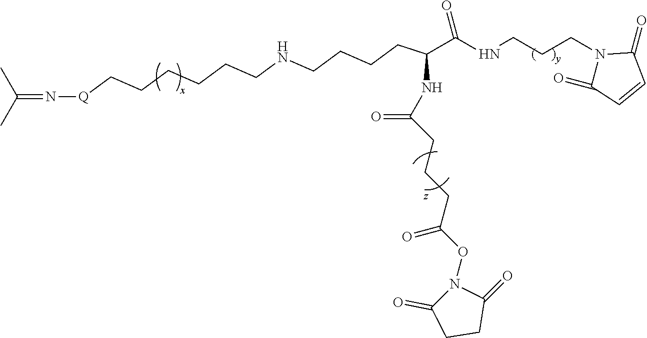

wherein x, y, z are independently an integer from 1 to 25 is provided.

In some embodiments, a compound of structure:

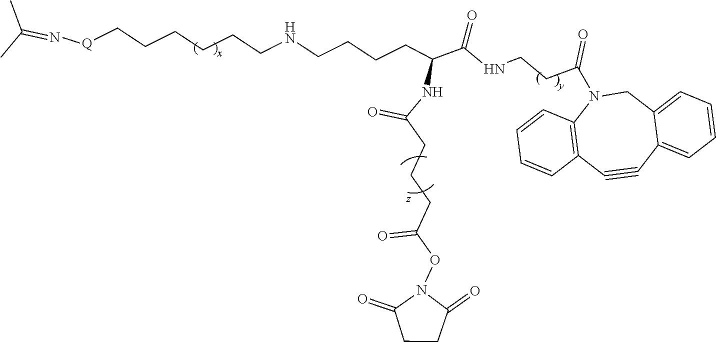

wherein x, y, z are independently an integer from 1 to 25 is provided.

In some embodiments, a compound of structure:

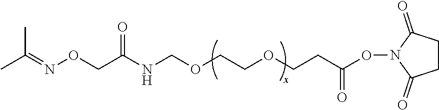

wherein x, y, z are independently an integer from 1 to 25 is provided.

In some embodiments, a compound of structure:

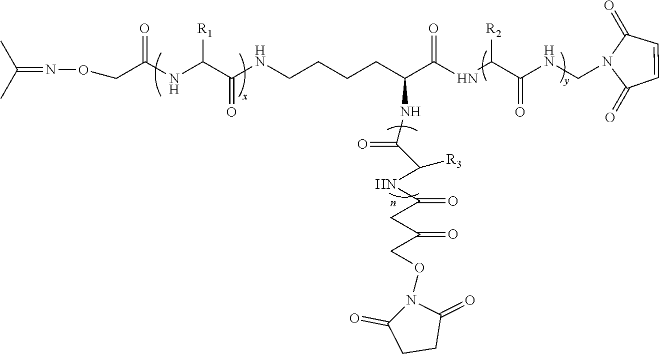

wherein R¹, R², R³ are independently hydrogen, alkyl or substituted alkyl, x, y, z are independently an integer from 1 to 25 is provided.

In some embodiments, a compound of structure:

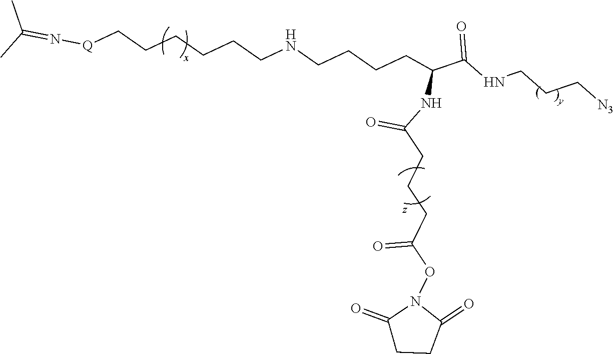

wherein R¹, R², R³ are independently hydrogen, alkyl or substituted alkyl, x, y, z are independently an integer from 1 to 25 is provided.

In some embodiments, a compound of structure:

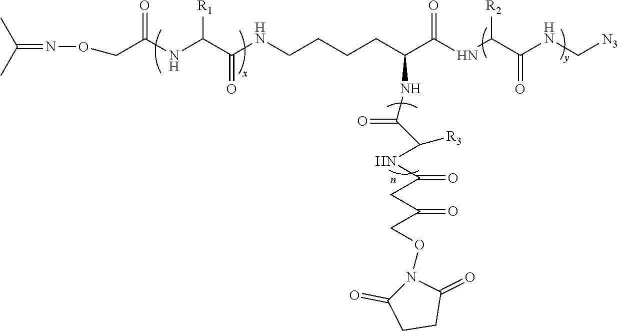

wherein R¹, R², R³ are independently hydrogen, alkyl or substituted alkyl, x, y, z are independently an integer from 1 to 25 is provided.

In some embodiments, a compound of structure:

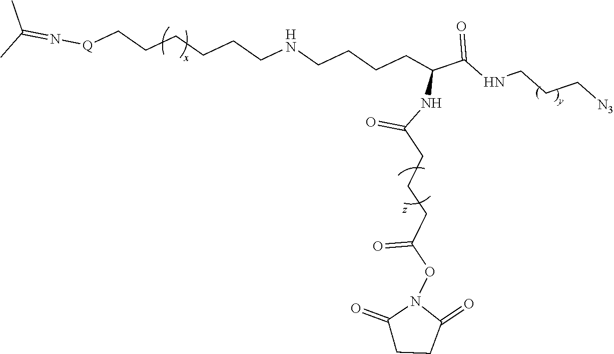

wherein R¹, R², R³ are independently hydrogen, alkyl or substituted alkyl, x, y, z are independently an integer from 1 to 25 is provided.

In some embodiments, a compound of structure:

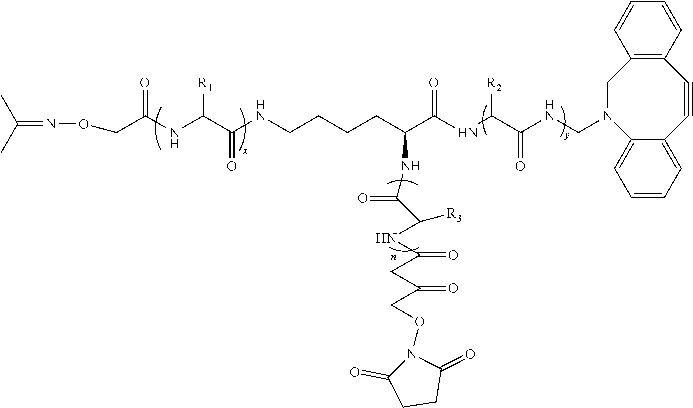

wherein R¹, R², R³ are independently hydrogen, alkyl or substituted alkyl, x, y, z are independently an integer from 1 to 25 is provided.

In some embodiments, a compound of structure:

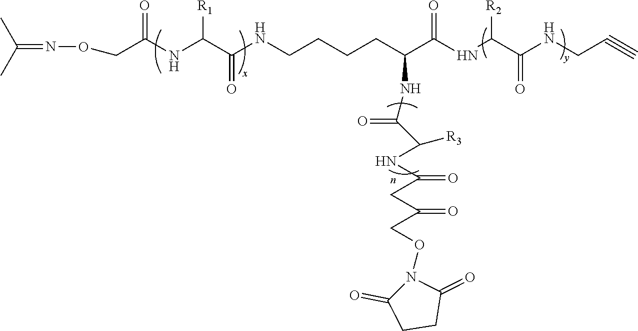

wherein R¹, R², R³ are independently hydrogen, alkyl or substituted alkyl, x, y, z are independently an integer from 1 to 25 is provided.

In some embodiments, compounds of structure:

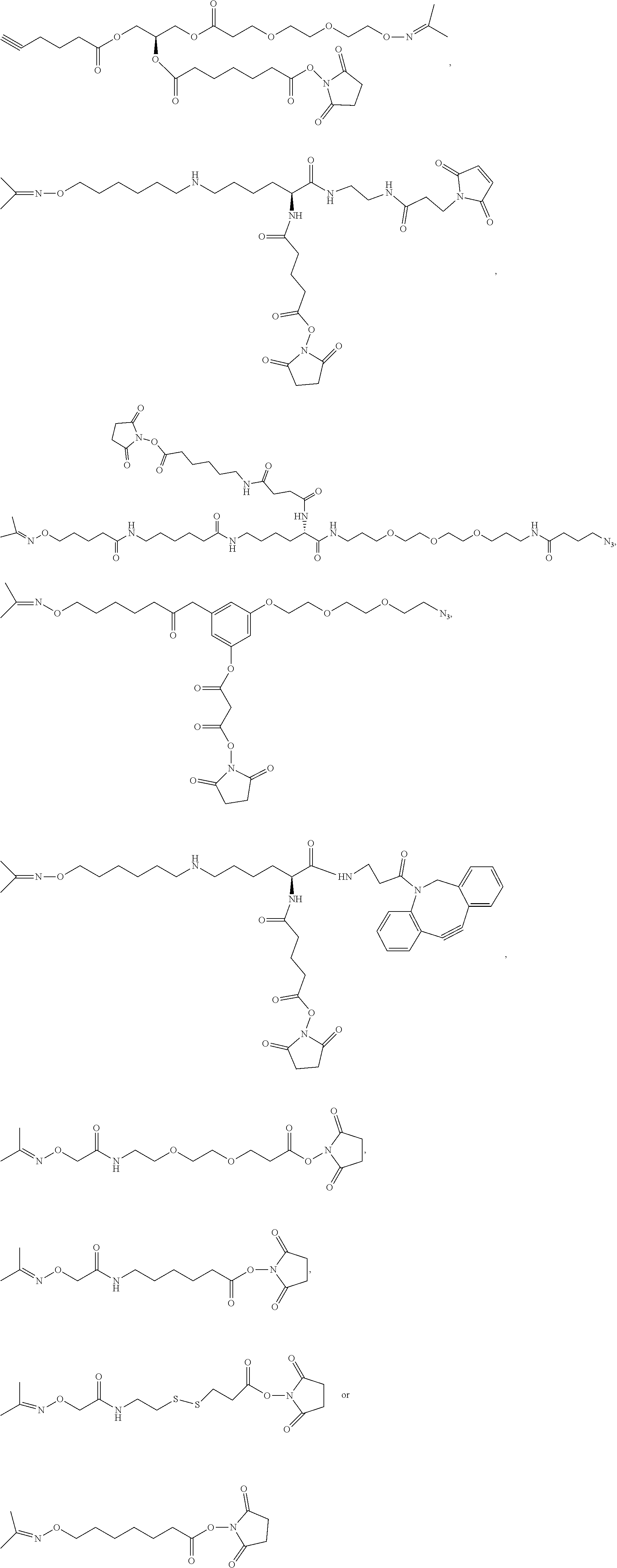

are provided.

### Methods of Using Crosslinkers

Illustrated below in Schemes 1-6 are some exemplary uses of the crosslinkers described above. The skilled artisan will comprehend that many other uses of the crosslinkers described herein exist, such as, for example conjugation of two nucleic acids, conjugation of a nucleic acid with a protein, conjugation of two drug molecules, conjugation of two lipids, conjugation of fluorophores with biomolecules, conjugation of dyes with biomolecules, conjugation of biomolecules with solid materials etc. and that the below description is not in any way limiting and includes conjugation of any two organic molecules. In general, the crosslinker described herein may be used biomolecules with each other, biomolecules with small molecules, small molecules with each other and biomolecules and/or small molecules with metal surfaces.

Scheme 1 illustrates formation of a magnetic particle functionalized with a free aminoxy group. Reaction of magnetic nanoparticles with a free amine group with crosslinkers of formula (I) results in formation of magnetic nanoparticles with attached amide groups which include terminal free aminooxy groups. The aminooxy groups may be used, for example to attach antibodies, proteins, nucleic acids, lipid, drugs, diagnostic agents, etc. to the amino magnetic nanoparticles.

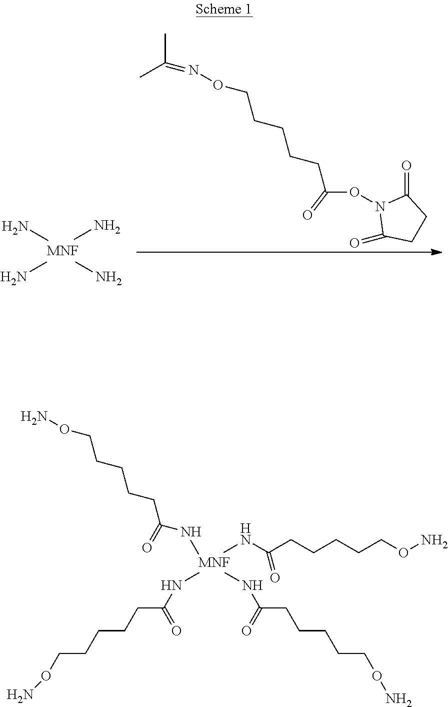

Scheme 2 illustrates the conjugation of two antibodies. Antibody 1, which includes a free amino group is reacted with a crosslinker of formula (I) to form an antibody with an amide functionalized with an free aminooxy group. Antibody 2 is oxidized to provide an antibody with an aldehyde group. Reaction of antibody 1 with a functionalized with an free aminooxy group with aldehyde 2 with a free aldehyde group results in formation of a stable oxime linkage which attaches antibody 1 to antibody 2.

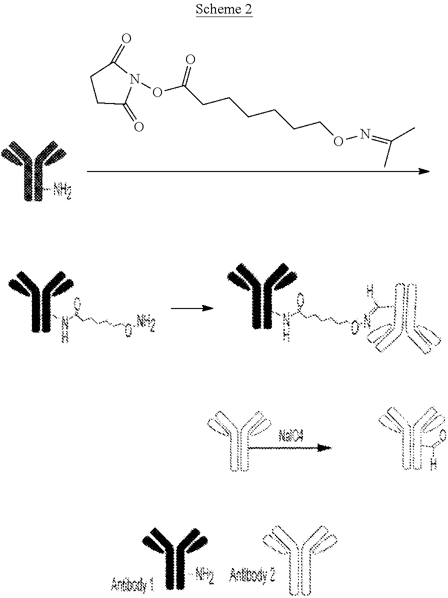 [SMILES: CC(C)=NOCCCCCCC(=O)ON1C(=O)CCC1=O]

Scheme 3 illustrates the conjugation of two antibody fragments. Antibody fragment 1, which includes a free amino group is reacted with a crosslinker of formula (I) to form an antibody fragment with an amide functionalized with an free aminooxy group. Antibody fragment 2 which also includes a free amino group is reacted with an NHS ester which contains a free aldehyde group. The aldehyde containing derivative of antibody fragment 2 is then reacted with antibody fragment 1, which includes an amide functionalized with an free aminooxy group to conjugate antibody fragment 1 with antibody fragment 2.

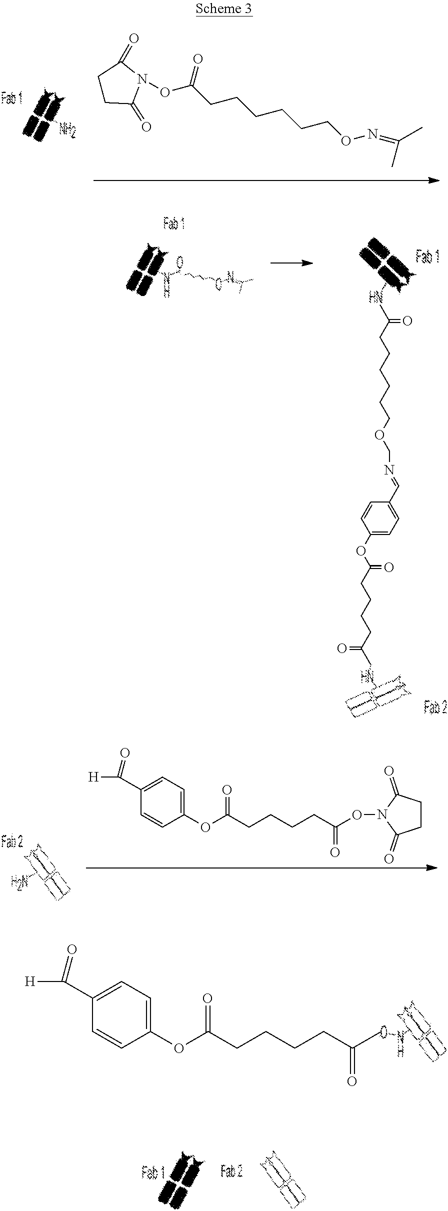 [SMILES: CC(C)=NOCCCCCCC(=O)ON1C(=O)CCC1=O ; CC(=O)CCCCCCOC/N=C/c1ccc(OC(=O)CCCCC(C)=O)cc1 ; O=Cc1ccc(OC(=O)CCCCC(=O)ON2C(=O)CCC2=O)cc1 ; CC(=O)CCCCC(=O)Oc1ccc(C=O)cc1]

Scheme 4 illustrates conjugation of a peptide drug to an antibody. A peptide drug, which includes a free alcohol group is reacted with a crosslinker of formula (I) to form an drug with an attached ester functionalized with an free aminooxy group. An antibody is oxidized to provide an antibody with an aldehyde group. The antibody with an aldehyde group is then reacted with the peptide drug which includes an alcohol functionalized with an free aminooxy group to form the conjugate of antibody with the peptide drug.

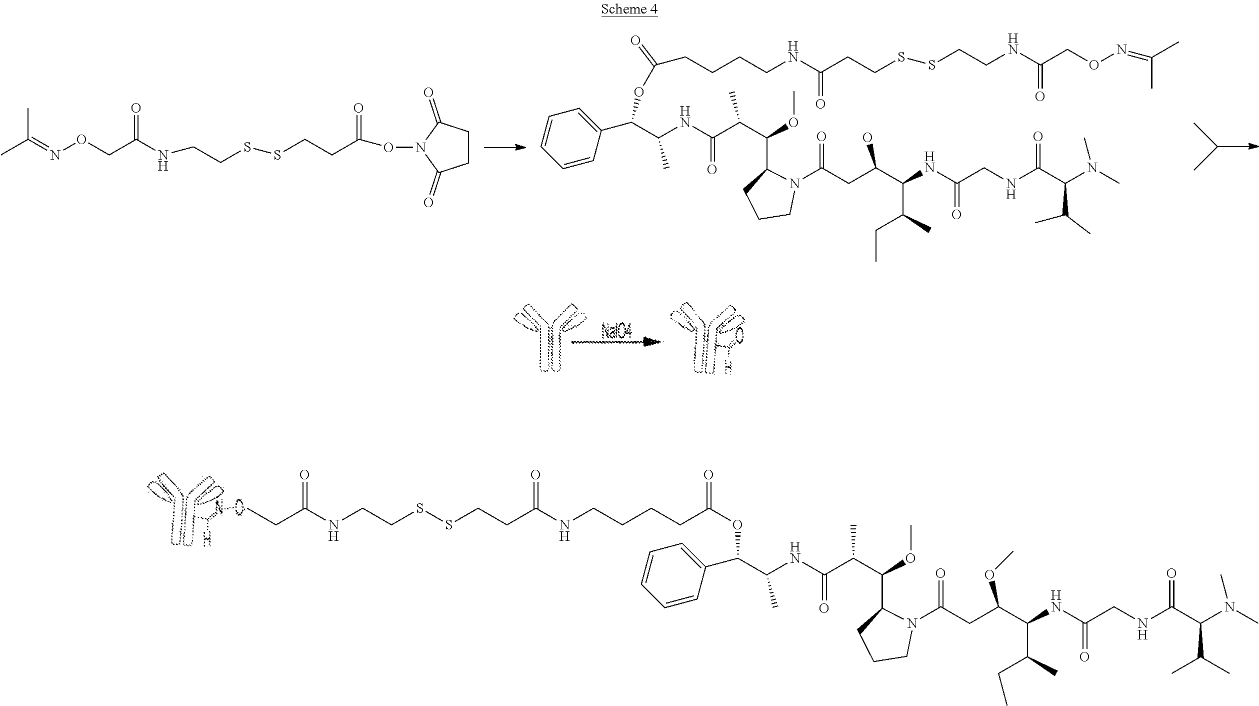

Scheme 5 illustrates the conjugation of a protein with an antibody. A protein which includes a free amino group is reacted with a crosslinker of formula (I) to form a protein with an attached amide functionalized with an free aminooxy group. An antibody which also includes a free amino group is reacted with an NHS ester which contains a free aldehyde group. The aldehyde containing antibody is then reacted with the protein which includes an amide functionalized with an free aminooxy group to conjugate the antibody to the protein.

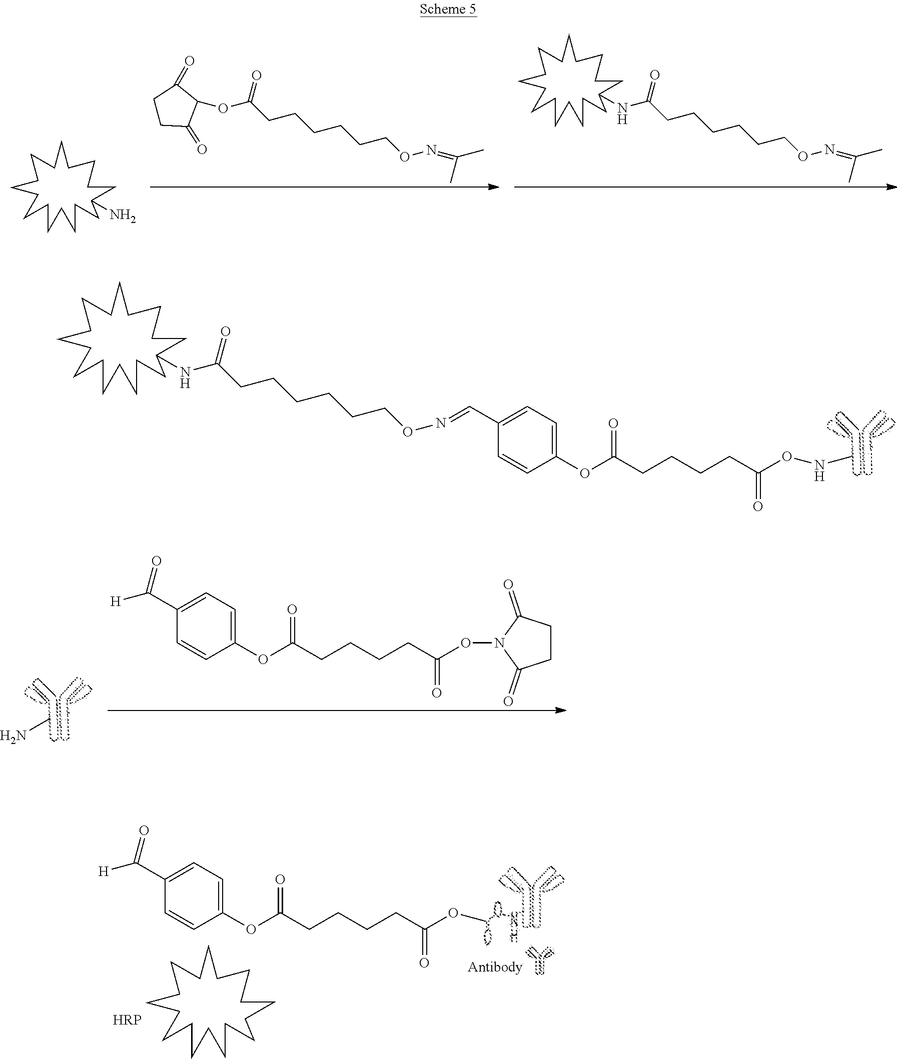

Scheme 6 illustrates the conjugation of an oligonucleotide with an antibody. An oligonucleotide which includes a free amino group is reacted with a crosslinker of formula (I) to form an nucleic acid amide functionalized with an free aminooxy group. An antibody which also includes a free amino group is reacted with an NHS ester which contains a free aldehyde group. The aldehyde containing antibody is then reacted with the oligonucleotide which includes an amide functionalized with an free aminooxy group to conjugate the nucleic acid to the protein.

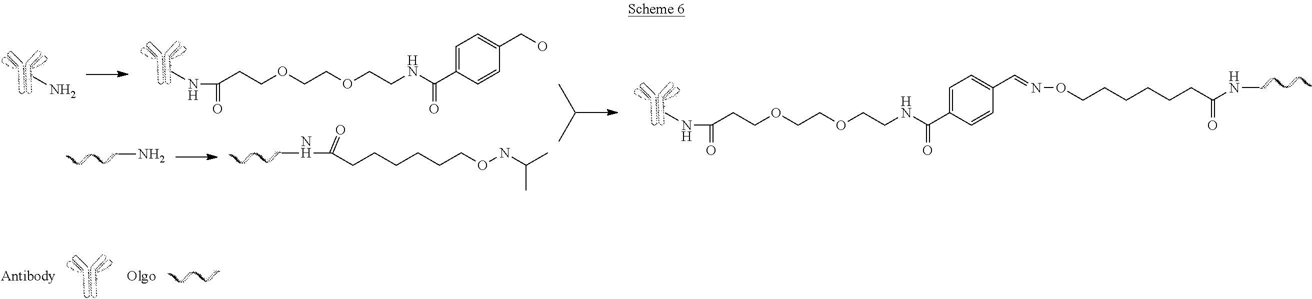

Exemplary antibodies include, but are not limited to, anti-HER2 antibody, anti-STEAP2 antibody, anti-MET antibody, anti-EGFRVIII antibody, anti-MUC 16 antibody, anti-PRLR antibody, anti-PSMA antibody, anti-FGFRα antibody, anti-FOLR1 antibody, anti-CD19, anti-BCMA, anti-TROP2, anti-Nectin-4, anti-CD79, anti-CD22, anti-CD30, antiCD33, antigen-binding fragment thereof, etc.

Exemplary drugs include, but are not limited to, doxorubicin, calicheamicin, monomethyl auristatin E, DM1 (derivative of maytansine), topoisomerase I inhibitor, PE38, MMAE (auristatin), Dxd (camptothecin), SG31999 (Pyrrolbenzodiazepine dimer), maytansinoid DM4, paclitaxel, SN-38, MMAF, etc.

Exemplary enzymes include, but are not limited to, HRP, Transglutaminase, Transpeptidase sortase A, ALP, etc.

Exemplary proteins include, but are not limited to, lipoproteins, glycoproteins, nucleoproteins, phosphoproteins, hemoproteins, flavoproteins, metalloproteins, phytochromes, cytochromes, opsins, and chromoproteins.

Exemplary lipids include, but are not limited to, cholesterol, beta-sitosterol, triglyceride, phospholipid, glycerophospholipid, Sphingolipids, polyketide, DOTAP, DOTMA, DSTAP, DPTAP, DMTAP, LPTAP, DLTAP, DORIT, DORIE-HP, DORIE-HB, DORIE-HPe, DORI, DMRIE, DPRIE, DSRIE, DODMA-SN, DSDAC, DODAC, DC-CHOL, DODMA, DODAP, DSPC, HSPC, Egg PC, DOPC, POPC, DOPE, C-DOPE, DAPE, DLPE, DPPE, POPE, DSPC, PEG-C-DMA, DSDMA, etc.

Exemplary antibody drug conjugates include but are not limited to, anti CD33 conjugated with MMAE, anti CD30 conjugated with MMAE, anti CD22 conjugated with MMAF, anti CD22 conjugated with PE38, anti HER2 conjugated with Dxd, anti HEG2 conjugated with SN38, anti NEctin-4 conjugated with MMAE, anti HER2 conjugated with MMAE, etc.

Finally, it should be noted that there are alternative ways of implementing the present invention. Accordingly, the present embodiments are to be considered as illustrative and not restrictive, and the invention is not to be limited to the details given herein but may be modified within the scope and equivalents of the appended claims. All publications and patents cited herein are incorporated by reference in their entirety.

### Examples

The following examples are intended only to illustrate the disclosure and are not intended to limit the scope of the invention. Other processes, assays, studies, protocols, procedures, methodologies, reagents and conditions may alternatively be used as appropriate.

As used herein, the symbols and conventions used in these processes, schemes, and examples, regardless of whether a particular abbreviation is specifically defined, are consistent with those used in the contemporary scientific literature, for example, the Journal of the American Chemical Society, the Journal of Medicinal Chemistry, or the Journal of Biological Chemistry.

The chemical reactions described in the Examples can be readily adapted to prepare a number of other compounds of the present disclosure, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by utilizing other suitable reagents known in the art other than those described, or by making routing modifications of reaction conditions, reagents, and starting materials.

Alternatively, other reactions disclosed herein or known in the art will be recognized as having applicability for preparing other compounds of the present disclosure.

### Definitions of Abbreviations

AcN Acetonitrile
AcOH Acetic acid
aq. Aqueous
Boc tert-butyloxycarbonyl
CSA Camphorsulfonic acid
CSCl₂ Thiophosgene
DCM Dichloromethane
DMF N,N′-Dimethylformamide
DMAP Dimethylaminopyridine
DIPEA Diisopropylethylamine
EDC 1-Ethyl-3-(3-dimethylaminopropyl) carbodiimide
DMSO Dimethylsulfoxide
Et Ethyl
Eq Equivalents
g Gram(s)
hr Hour (hours)
HCl Hydrochloric acid
HPLC High-performance liquid chromatography
LC/MS Liquid chromatography-mass spectrometry
Me Methyl
mg Milligrams
MeOH Methanol
mL Milliliter(s)
μl Microliter(s)
mol Moles
mmol Millimoles
μmol/umol Micromoles
MS Mass spectrometry
r.t. Room temperature
t-Bu tert-Butyl
TEA or Et₃N Triethylamine
TFA Trifluoracetic acid
THF Tetrahydrofuran
TLC Thin Layer Chromatography
NHS N-hydroxy succinimide
ADC Antibody-drug conjugate
aq. Aqueous

Most starting materials were commercially available from Sigma-Aldrich, J&K, Chemexpress, etc.

Scheme 7 illustrates the preparation of compound 1.

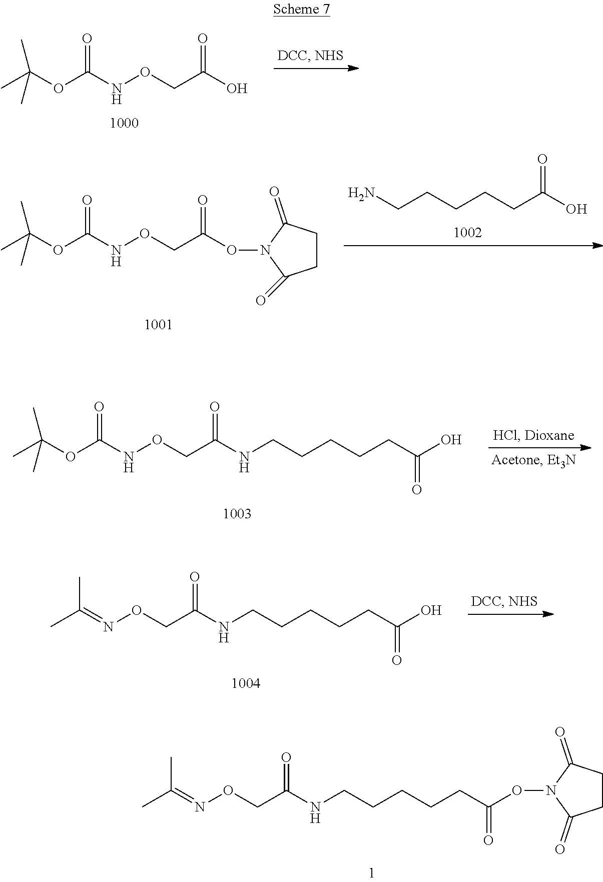 [SMILES: CC(C)(C)OC(=O)NOCC(=O)O ; CC(C)(C)OC(=O)NOCC(=O)ON1C(=O)CCC1=O ; NCCCCCC(=O)O ; CC(C)(C)OC(=O)NOCC(=O)NCCCCCC(=O)O ; CC(C)=NOCC(=O)NCCCCCC(=O)O ; CC(C)=NOCC(=O)NCCCCCC(=O)ON1C(=O)CCC1=O]

### Preparation of Compound 1001

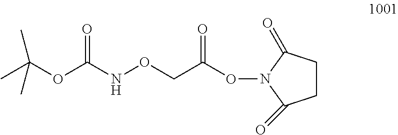 [SMILES: CC(C)(C)OC(=O)NOCC(=O)ON1C(=O)CCC1=O]

To a mixture of compound 1000 (10.00 g, 52.3 mmol) in anhydrous DCM (100 mL), DCC (21.5 g, 2 eq) and NHS (7.2 g, 1.2 eq) was added. The resulting solution was stirred at r.t. overnight to provide compound 1001 (13.1 g) which was purified by silica gel chromatography (ethyl acetate:Methanol=3:1). ESI-MS (m/z): calculated for C₁₁H₁₆N₂O₇, 288.26, found [M+H⁺] 289.5.

### Preparation of Compound 1003

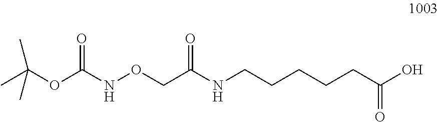 [SMILES: CC(C)(C)OC(=O)NOCC(=O)NCCCCCC(=O)O]

6-amino hexanoic acid (1002) (1.0 g, 7.6 mmol) was added to compound 1001 (2.5 g, 8.7 mmol) in anhydrous DCM (20 ml). The resulting solution was stirred at r.t. for 6 hours to provide compound 1003 (1.85 g) which was purified by silica gel chromatography (Ethyl acetate:Methanol=5:1). ESI-MS (m/z): calculated for C₁₃H₂₄N₂O₆, 304.34, found [M−H⁺] 303.4.

### Preparation of Compound 1004

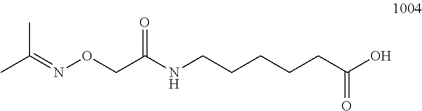 [SMILES: CC(C)=NOCC(=O)NCCCCCC(=O)O]

Compound 1003 (500 mg, 1.6 mmol) was dissolved in hydrochloric acid in dioxane (50 ml). The resulting mixture was stirred at r.t. for 30 min followed by concentration. Then the reaction mixture was dissolved in acetone (20 ml), and Et₃N (1 ml) was added to the mixture and the reaction mixture was stirred at rt overnight to provide compound 1004 (320 mg) which was purified through silica gel chromatography (ethyl acetate:Methanol=2:1). ESI-MS (m/z): calculated for C₁₁H₂₀N₂O₄, 244.29 found [M−H⁺] 243.4.

### Example 1: Preparation of Compound 1

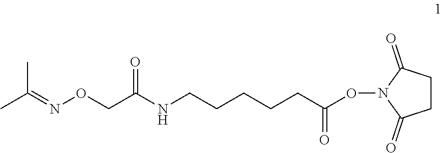 [SMILES: CC(C)=NOCC(=O)NCCCCCC(=O)ON1C(=O)CCC1=O]

Compound 1004 (320 mg, 0.94 mmol) was dissolved in anhydrous DCM (20 ml), DCC (390 mg, 2 eq) and NHS (130 mg, 1.2 eq) were added and the solution was stirred at r.t. for another 4 h to provide compound 1 (256 mg) which was purified by silica gel chromatography (Hexanes:Ethyl acetate=1:1). ¹HNMR (500 MHZ, CDCl₃): δ, 1.33 (m, 2H), 1.54-1.50 (m, 4H), 2.30 (m, 2H), 2.64 (m, 4H), 2.99 (s, 6h), 3.02 (m, 2H), 4.87 (s, 2H0, 8.01 (s, 1H).

Scheme 8 illustrates the preparation of compound 2.

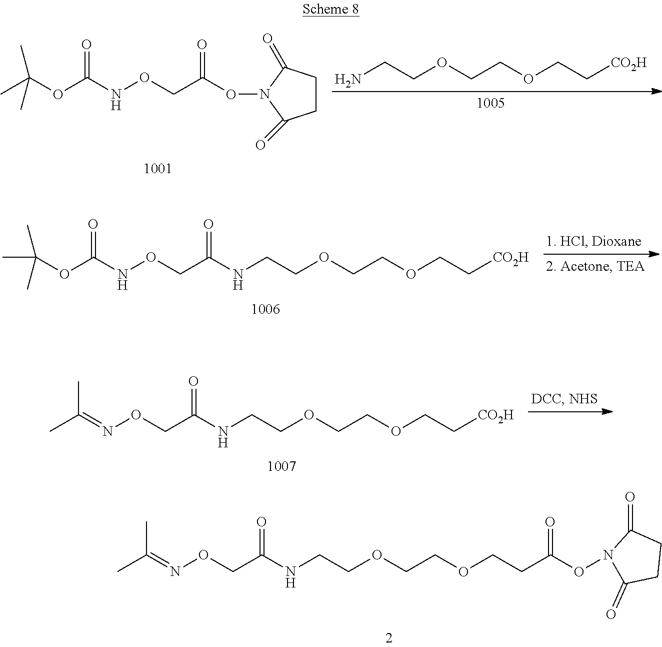

### Preparation of Compound 1006

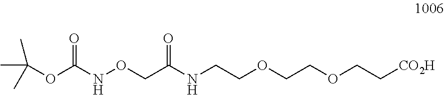 [SMILES: CC(C)(C)OC(=O)NOCC(=O)NCCOCCOCCC(=O)O]

Compound 1005 (1.55 g, 8.7 mmol) was added to compound 1001 (2.5 g, 8.7 mmol) in anhydrous DCM (20 ml). The resulting solution was stirred at r.t. for 6 hours to provide compound 1006 (2.28 g) which was purified by silica gel chromatography (Ethyl acetate:Methanol=3:1). ESI-MS (m/z): calculated for C₁₄H₂₆N₂O₈, 350.37, found [M−H⁺] 349.5.

### Preparation of Compound 1007

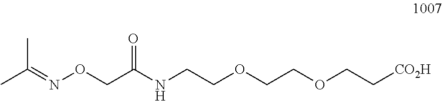 [SMILES: CC(C)=NOCC(=O)NCCOCCOCCC(=O)O]

A solution of compound 1006 (1.75 mg, 5.0 mmol was dissolved in hydrochloric acid in dioxane (50 ml). The resulting mixture was stirred at r.t. for 30 min followed by concentration by rotary evaporation. Then the reaction mixture was dissolved in acetone (20 ml), and Et₃N (1 ml) was added and the reaction mixture was stirred at r.t. overnight to provide compound 1007 (725 mg) which was purified by silica gel chromatography (Ethyl acetate:Methanol=2:1). ESI-MS (m/z): calculated for C₁₂H₂₂N₂O₆, 290.32, found [M−H⁺] 289.4.

### Example 2: Preparation of Compound 2

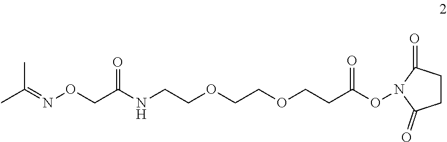 [SMILES: CC(C)=NOCC(=O)NCCOCCOCCC(=O)ON1C(=O)CCC1=O]

Compound 1007 (500 mg, 1.72 mmol) was dissolved in anhydrous DCM (20 ml), DCC (710 mg, 2 eq) and NHS (240 mg, 1.2 eq) were added, and the solution was stirred at r.t. for another 4 h to provide compound 2 (450 mg) which was purified by silica gel chromatography (Ethyl acetate:Methanol=2:1). ¹HNMR (500 MHZ, CDCl₃): δ, 2.40 (m, 2H), 2.64 (m, 4H), 2.99 (s, 6H), 3.02 (m, 2H), 3.52-3.67 (m, 8H), 4.87 (s, 2H), 6.39 (s, 1H), 8.01 (s, 1H).

Scheme 9 illustrates the preparation of compound 3.

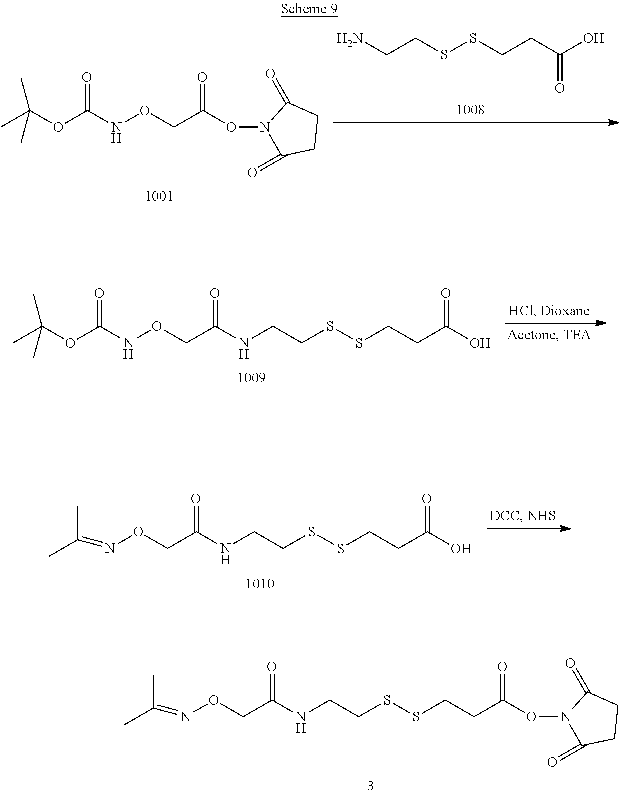 [SMILES: CC(C)(C)OC(=O)NOCC(=O)ON1C(=O)CCC1=O ; NCCSSCCC(=O)O ; CC(C)(C)OC(=O)NOCC(=O)NCCSSCCC(=O)O ; CC(C)=NOCC(=O)NCCSSCCC(=O)O ; CC(C)=NOCC(=O)NCCSSCCC(=O)ON1C(=O)CCC1=O]

### Preparation of Compound 1009

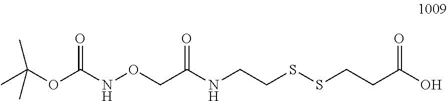 [SMILES: CC(C)(C)OC(=O)NOCC(=O)NCCSSCCC(=O)O]

Compound 1008 (1.57 g, 8.7 mmol) was added to compound 1001 (2.5 g, 8.7 mmol) in anhydrous DCM (20 ml). The resulting solution was stirred at r.t. for 6 hours to provide compound 1009 (2.03 g) which was purified by silica gel chromatography (Ethyl acetate:Methanol=3:1). ESI-MS (m/z): calculated for C₁₂H₂₂N₂O₆S₂, 354.44, found [M−H⁺] 353.3.

### Preparation of Compound 1010

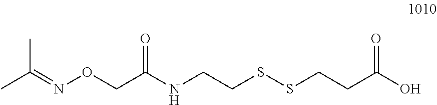 [SMILES: CC(C)=NOCC(=O)NCCSSCCC(=O)O]

A solution of compound 1009 (1.75 g, 5.0 mmol) was dissolved in hydrochloric acid in dioxane (50 ml). The resulting mixture was stirred at r.t. for 30 min followed by concentration by rotary evaporation. The reaction mixture was dissolved in acetone (20 ml), and Et₃N (1 ml) was added and the reaction mixture was stirred at r.t. overnight to provide compound 1010 (725 mg). ESI-MS (m/z): calculated for C₁₀H₁₈N₂O₄S₂, 294.07, found [M−H⁺] 293.2.

### Example 3: Preparation of Compound 3

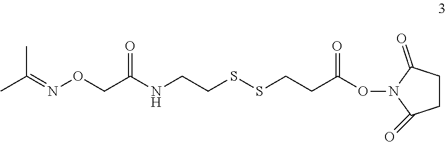 [SMILES: CC(C)=NOCC(=O)NCCSSCCC(=O)ON1C(=O)CCC1=O]

Compound 1009 (500 mg, 1.72 mmol) was dissolved in anhydrous DCM (20 ml), DCC (680 mg, 2 eq) and NHS (220 mg, 1.2 eq) were added and the solution was stirred at r.t. for another 4 h. Compound 3 (370 mg) was obtained by silica gel chromatography (Hexanes:Ethyl acetate=1:2). ¹HNMR (500 MHZ, CDCl₃): δ, 2.40 (m, 2H), 2.64 (m, 4H), 2.85 (s, 4H), 2.98 (S, 6H), 3.52-3.67 (m, 2H), 4.87 (s, 2H), 8.00 (s, 1H).

Scheme 10 illustrates the preparation of compound 4.

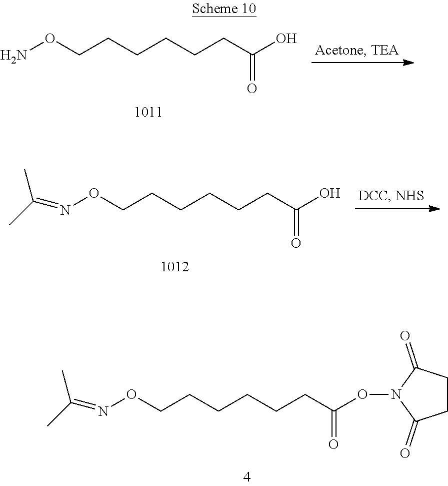 [SMILES: NOCCCCCCC(=O)O ; CC(C)=NOCCCCCCC(=O)O ; CC(C)=NOCCCCCCC(=O)ON1C(=O)CCC1=O]

### Preparation of Compound 1012

Compound 1011 (500 mg, 5.0 mmol) was dissolved in acetone (20 ml), and Et₃N (1 ml) was added and the reaction mixture was stirred at r.t. overnight to provide compound 1012 (500 mg) which was not further purified.

### Example 4: Preparation of Compound 4

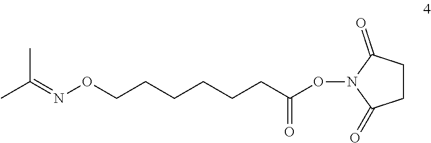 [SMILES: CC(C)=NOCCCCCCC(=O)ON1C(=O)CCC1=O]

Compound 1012 (50 mg, 5.0 mmol) was dissolved in anhydrous DCM (20 ml) and DCC (2.06 g, 2 eq) and NHS (690 mg, 1.2 eq) were added and the solution was stirred at r.t. for another 4 h to provide compound 4 (370 mg) which was purified by silica gel chromatography (Hexanes:Ethyl acetate=1:1). ¹HNMR (500 MHz, CDCl₃): δ, 1.30-1.52 (m, 8H), 2.40 (m, 2H), 3.51 (m, 2H), 2.98 (S, 6H), 3.52-3.67 (m, 2H).

Scheme 11 illustrates the preparation of compound 5.

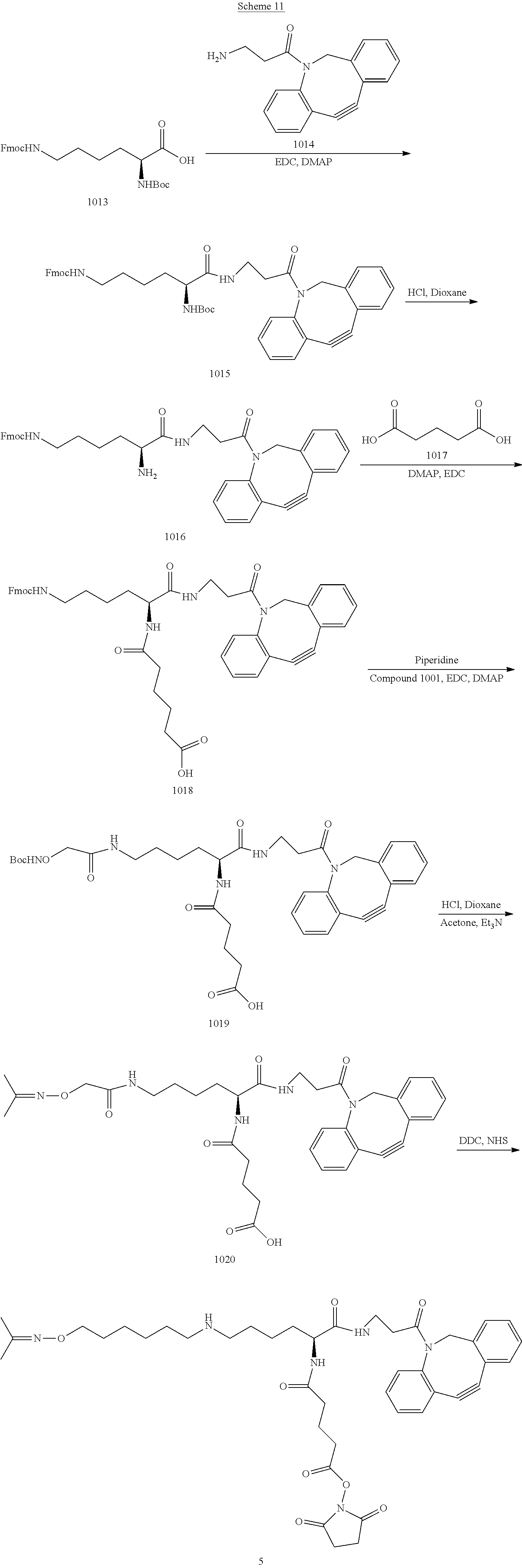

### Preparation of Compound 1015

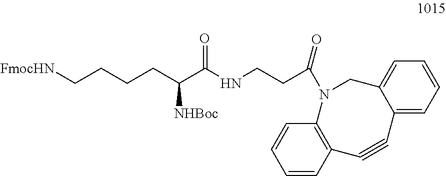

Compound 1013 (9.37 g, 20 mmol) was added to compound 1014 (5.52 g, 20 mmol) in the presence of EDC (5.75 g, 30 mmol and DMAP (2.76 g, 30 mmol) in anhydrous DCM (100 ml) and the resulting solution was stirred at r.t. for 6 hours to provide compound 1015 (8.72 g) which was purified by silica gel chromatography (Hexanes:Ethyl acetate=1:2). ESI-MS (m/z): calculated for C₄₄H₄₆N₄O₆, 726.87, found [M+H⁺] 727.9.

### Preparation of Compound 1016

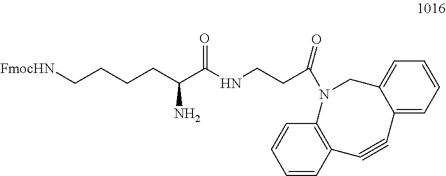

A solution of compound 1015 (5.0 g, 6.9 mmol) was dissolved in hydrochloric acid in dioxane (50 ml) and the reaction mixture was stirred at r.t. for 2 hours. Compound 1016 (2.16 g) was obtained by silica gel chromatography (Ethyl acetate:Methanol=1:1). ESI-MS (m/z): calculated for C₃₉H₃₈N₄O₄, 626.76, found [M+H⁺] 627.5.

### Preparation of Compound 1018

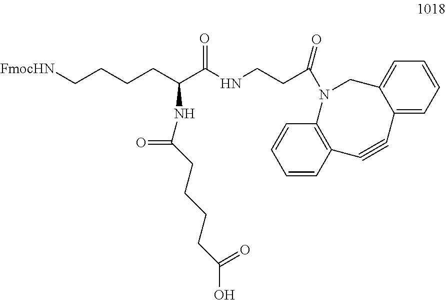

Compound 1016 (2.16 g, 3.5 mmol) was dissolved in anhydrous DCM (20 ml) and compound 1017 (330 mg, 0.8 eq), EDC (1.34 g, 2 eq) and NHS (600 mg, 12 eq) were added and the solution was stirred at rt for another 4 h. Compound 1018 (1.32 g, 1.78 mmol) was obtained by silica gel chromatography (Ethyl acetate:Methanol=3:1). ESI-MS (m/z): calculated for C₄₄H₄₄N₄O₇, 740.32, found [M−H⁺] 739.5.

### Preparation of Compound 1019

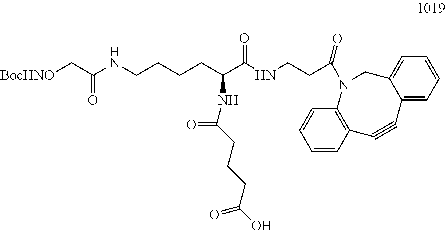

A solution of compound 1018 (3.77 g, 5.0 mmol) was dissolved in anhydrous DCM (50 ml) and piperidine (10 ml) was added to remove the Fmoc protecting group. The deprotected group was extracted, washed then directed used for further step. Compound 1001 (2.62 g), EDC (1.09 g, 2 eq) and DMAP (915 mg, 1.5 eq) were added to the crude free amine compound DCM solution to provide compound 1019 (2.62 g) which was purified by silica gel chromatography (Ethyl acetate:Methanol=1:1). ESI-MS (m/z): calculated for C₃₆H₄₅N₅O₉, 691.78, found [M−H⁺] 691.0.

### Preparation of Compound 1020

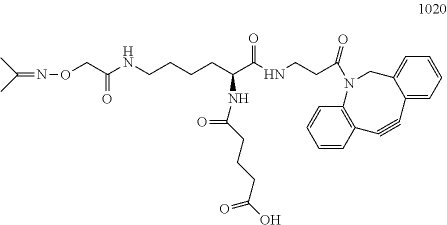 [SMILES: CC(C)=NOCC(=O)NCCCC[C@H](NC(=O)CCCC(=O)O)C(=O)NCCC(=O)N1Cc2ccccc2C#Cc2ccccc21]

A solution of compound 1019 (1.39 g, 2.0 mmol) was dissolved in hydrochloric acid in dioxane (50 ml). The resulting mixture was stirred at r.t. for 30 min followed by concentration by rotary evaporation. Then, the reaction mixture was dissolved in acetone (20 ml), and Et₃N (1 ml) was added and the reaction mixture was stirred at r.t. overnight to provide compound 1020 (757 mg) which was purified by silica gel chromatography. (Ethyl acetate:Methanol=1:1). ESI-MS (m/z): calculated. for C₃₄H₄₁N₅O₇, 631.73, found [M−H⁺] 630.9.

### Example 5: Preparation of Compound 5

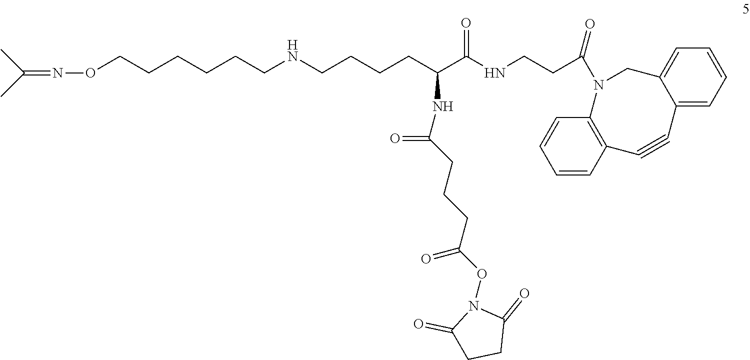 [SMILES: CC(C)=NOCCCCCCNCCCC[C@H](NC(=O)CCCC(=O)ON1C(=O)CCC1=O)C(=O)NCCC(=O)N1Cc2ccccc2C#Cc2ccccc21]

Compound 1020 (500 mg, 0.79 mmol) was dissolved in anhydrous DCM (20 ml) and DCC (410 mg, 2 eq) and NHS (137 mg, 1.5 eq) were added and the solution was stirred at r.t. for another 4 h to provide compound 5 (370 mg) which was purified by silica gel chromatography. ¹HNMR (500 MHz, CDCl₃): δ, 1.38-1.77 (m, 14H), 2.40 (m, 2H), 2.53 (m, 4H), 2.64 (m, 4H), 2.85 (s, 4H), 2.98 (S, 6H), 3.52-3.67 (m, 2H), 4.44 (m, 2H), 4.64 (s, 2H), 4.87 (s, 2H), 7.07-7.45 (m, 8H), 8.00 (s, 1H), 8.30 (s, 1H).

1. A compound of Formula (I): 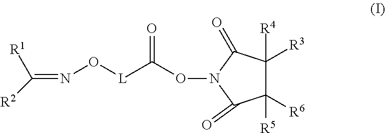
or salts, hydrate or solvates thereof wherein:
R¹ and R² are independently alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, heterocycloalkyl, substituted heterocycloalkyl, heteroalkyl, substituted heteroalkyl, heteroalkenyl, substituted heteroalkenyl, heteroaryl, substituted heteroaryl, heteroarylalkyl, substituted heteroarylalkyl, heteroarylalkenyl, substituted heteroarylalkenyl, or together with the atoms to which they are bonded form a cycloalkyl, substituted cycloalkyl, cycloalkenyl, substituted cycloalkenyl, heterocycloalkyl, substituted heterocycloalkyl, heterocycloalkenyl or substituted heterocycloalkenyl ring;
R³, R⁴, R⁵ and R⁶ are independently —H, alkyl, substituted alkyl, alkenyl, substituted alkenyl, aryl, substituted aryl, arylalkyl, substituted arylalkyl, arylalkenyl, substituted arylalkenyl, cycloalkyl, substituted cycloalkyl, heterocycloalkyl, substituted heterocycloalkyl, heteroalkyl, substituted heteroalkyl, heteroalkenyl, substituted heteroalkenyl, heteroaryl, substituted heteroaryl, heteroarylalkyl, substituted heteroarylalkyl, heteroarylalkenyl, substituted heteroarylalkenyl or optionally one of R³, R⁴, R⁵ and R⁶ is —SO₃H or a salt thereof;
L is alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl, substituted heteroarylalkenyldiyl, a complementary oligonucleotide between 1 and 30 nucleic acid bases, a complementary oligopeptide between 1 and 30 amino acids or -L₁X(L₂Z)ₐL₃-;
L₁, L₂ and L₃ are independently alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl or substituted heteroarylalkenyldiyl;
X is alkyldiyl, substituted alkyldiyl, alkenyldiyl, substituted alkenyldiyl, aryldiyl, substituted aryldiyl, arylalkyldiyl, substituted arylalkyldiyl, arylalkenyldiyl, substituted arylalkenyldiyl, cycloalkyldiyl, substituted cycloalkyldiyl, heterocycloalkyldiyl, substituted heterocycloalkyldiyl, heteroalkyldiyl, substituted heteroalkyldiyl, heteroalkenyldiyl, substituted heteroalkenyldiyl, heteroaryldiyl, substituted heteroaryldiyl, heteroarylalkyldiyl, substituted heteroarylalkyldiyl, heteroarylalkenyldiyl, substituted heteroarylalkenyldiyl, alkyltriyl, heteroalkyltriyl, substituted heteroalkyltriyl, a complementary oligopeptide between 1 and 30 amino acids, a complementary oligonucleotide between 1 and 30 nucleic acid bases,
—(CH₂)ᵣS—S(CH₂)ᵣ—,
—(CH₂)ᵣ(O—CH₂CH₂)ₛ(CH₂)ᵣ—,
—(CH₂)ᵣC(O)(CH₂)ᵣ—,
—(CH₂)ᵣC(O)₂ (CH₂)ᵣ—,
—(CH₂)ᵣS(CH₂)ᵣ— or
—(CH₂)ᵣSO₂ (CH₂)ᵣ—;
each r is independently an integer between 3 and 25;
Z is maleimide, Texas Red, Alexa Fluor 48, Fluorescein isothiocyanate, Pyridine disulfide, Rhodamine, Rhodamine B, Tetramethyl Rhodamine, N₃, alkyne, 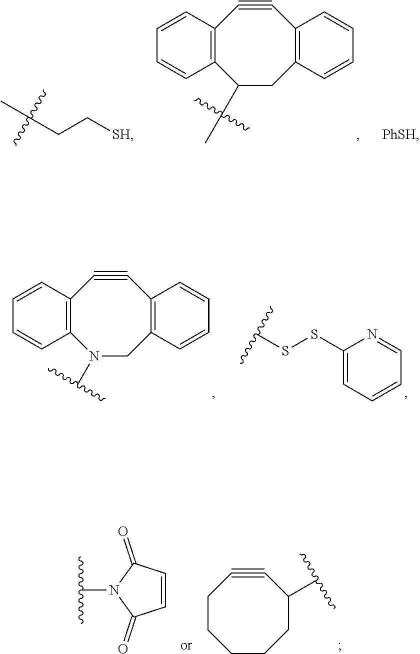
and a is 0 or 1.

2. The compound of claim 1, wherein R¹ and R² are independently alkyl, alkenyl, aryl, or arylalkyl.

3. The compound of claim 1, wherein R¹ and R² are independently —CH₃, —C₂H₅, —C₃H₇, cyclopentyl, cyclohexyl, allyl, phenyl or benzyl.

4. The compound of claim 1, wherein R¹ and R² are independently —CH₃, —C₂H₅, or phenyl.

5. The compound of claim 1, wherein R³, R⁴, R⁵ and R⁶ are independently —H, alkyl, alkenyl, aryl, arylalkyl or —SO₃H or a salt thereof.

6. The compound of claim 1, wherein R³, R⁴, R⁵ and R⁶ are independently —H, —CH₃, —C₂H₅, —C₃H₇, allyl, phenyl, benzyl or —SO₃H or a salt thereof.

7. The compound of claim 1, wherein R³, R⁴, R⁵ and R⁶ are independently —H, —CH₃ or —SO₃H or a salt thereof.

8. The compound of claim 1, wherein L is 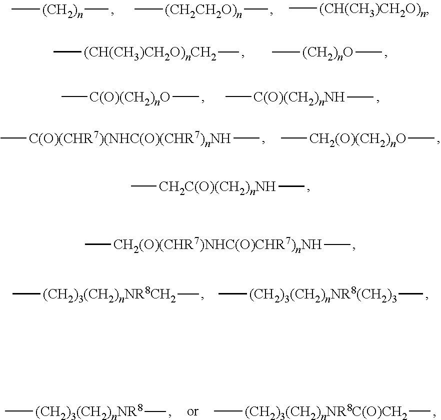
a complementary oligonucleotide between 1 and 30 nucleic acid bases;
a complementary oligopeptide between 1 and 30 amino acids;
each R⁷ is independently —H, (C₁-C₅) alkyl, (C₁-C₅) substituted alkyl, aryl, substituted aryl, arylalkyl, substituted arylalkyl, heteroarylalkyl or heteroaryl;
R⁸ is —H or alkyl; and
n is an integer between 1 and 30.

9. The compound of claim 1, wherein a is 0, L is L₁-X-L₃ and L₁ and L₃ are independently
—(CH₂)ₒC(O)OXOC(O)(CH₂)ₒ—,
—(CH₂)ₒC(O)NHXNHC(O)(CH₂)ₒ—,
—(CH₂)ₒX(CH₂)ₒ—,
—(CH₂)ₒX(CH₂)ₒO—,
—(CH₂)ₒX(CH₂)ₒNH—,
—C(O)(CH₂)ₒX(CH₂)ₒO—,
—C(O)(CH₂)ₒX(CH₂)ₒNH—,
—(CH₂)₂C(O)NHXNHC(O)(CH₂)₂—,
—(CH₂)₂C(O)NHXC(O)NH(CH₂)₂—,
—(CH₂)₂C(O)NHXOC(O)(CH₂)₂—,
—(CH₂)₂C(O)NHX(O)O(CH₂)₂—,
or —(CH₂)₂C(O)OXOC(O)(CH₂)₂—; X is alkyldiyl, substituted alkyldiyl, alkenyldiyl, substituted alkenyldiyl, aryldiyl, substituted aryldiyl, cycloalkyldiyl, substituted cycloheteroalkyldiyl, cycloheteroalkyldiyl, substituted cycloalkyldiyl, alkenyldiyl, substituted alkenyldiyl, heteroalkyldiyl, substituted heteroalkyldiyl, heteroalkenyldiyl, substituted heteroalkenyldiyl, heteroaryldiyl, substituted heteroaryldiyl; and o is an integer between 1 and 15.

10. The compound of claim 1, wherein a is 1, L is L₁-X(L₂Z)-L₃; L₁, L₂ and L₃ are independently —(CH₂)ₙ—
—(CH₂CH₂O)ₙ—,
—(CH₂)ₒC(O)OXOC(O)(CH₂)ₒ—,
—(CH₂)ₒC(O)NHXNHC(O)(CH₂)ₒ—,
—(CH₂)ₒX(CH₂)ₒ—,
—(CH₂)ₒX(CH₂)ₒO—,
—(CH₂)ₒX(CH₂)ₒNH—,
—C(O)(CH₂)ₒX(CH₂)ₒO—,
—C(O)(CH₂)ₒX(CH₂)ₒNH—,
—(CH₂)₂C(O)NHXNHC(O)(CH₂)₂—,
—(CH₂)₂C(O)NHXC(O)NH(CH₂)₂—
—(CH₂)₂C(O)NHXOC(O)(CH₂)₂—,
—(CH₂)₂C(O)NHX(O)O(CH₂)₂—,
—(CH₂)₂C(O)OXOC(O)(CH₂)₂—; a complementary oligopeptide between 1 and 30 amino acids; a complementary oligonucleotide between 1 and 30 nucleic acid bases; X is alkyltriyl, heteroalkyltriyl, substituted heteroalkyltriyl; and
o is an integer between 1 and 15.

11. The compound of claim 1, wherein L₁, L₂, and L₃ are independently C₁-C₂₀ alkyl, phenyl, aryl, —NH—, —C(O)—, —(CH₂)ₙ—NH—C(O)—, —(CH₂)ₙ—C(O)—NH—, —(CH₂—CH₂—O)ₙ—, —(O—CH₂—CH(CH₃))ₙ—C(O)—NH—, or —(OCH₂—CH(CH₃))ₙ—; and each n is independently an integer from 1 to 25.

12. The compound of claim 1, wherein R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof and L is 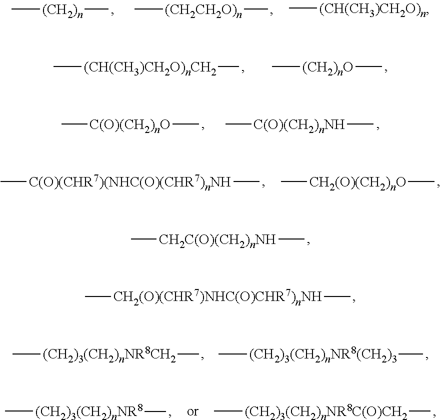
a complementary oligonucleotide between 1 and 30 nucleic acid bases, a complementary oligopeptide between 1 and 30 amino acids;
each R⁷ is independently —H, (C₁-C₅) alkyl, (C₁-C₅) substituted alkyl, aryl, substituted aryl, arylalkyl, substituted arylalkyl, heteroarylalkyl or heteroaryl;
R⁸ is —H or alkyl; and
n is an integer between 1 and 30.

13. The compound of claim 13, wherein R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

14. The compound of claim 1, wherein R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof, a is 0, L is L₁-X-L₃, L₁ and L₃ are independently
—(CH₂)ₒC(O)OXOC(O)(CH₂)ₒ—,
—(CH₂)ₒC(O)NHXNHC(O)(CH₂)ₒ—,
—(CH₂)ₒX(CH₂)ₒ—,
—(CH₂)ₒX(CH₂)ₒO—,
—(CH₂)ₒX(CH₂)ₒNH—,
—C(O)(CH₂)ₒX(CH₂)ₒO—,
—C(O)(CH₂)ₒX(CH₂)ₒNH—,
—(CH₂)₂C(O)NHXNHC(O)(CH₂)₂—,
—(CH₂)₂C(O)NHXC(O)NH(CH₂)₂—,
—(CH₂)₂C(O)NHXOC(O)(CH₂)₂—,
—(CH₂)₂C(O)NHX(O)O(CH₂)₂—,
or —(CH₂)₂C(O)OXOC(O)(CH₂)₂—; X is alkyldiyl, substituted alkyldiyl, alkenyldiyl, substituted alkenyldiyl, aryldiyl, substituted aryldiyl, cycloalkyldiyl, substituted cycloheteroalkyldiyl, cycloheteroalkyldiyl, substituted cycloalkyldiyl, alkenyldiyl, substituted alkenyldiyl, heteroalkyldiyl, substituted heteroalkyldiyl, heteroalkenyldiyl, substituted heteroalkenyldiyl, heteroaryldiyl, substituted heteroaryldiyl; and o is an integer between 1 and 15.

15. The compound of claim 15, wherein R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

16. The compound of claim 1, wherein R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof, a is 1, L is L₁-X(L₂Z)-L₃ and L₁, L₂ and L₃ are independently —(CH₂)ₙ—,
—(CH₂CH₂O)ₙ—,
—(CH₂)ₒC(O)OXOC(O)(CH₂)ₒ—,
—(CH₂)ₒC(O)NHXNHC(O)(CH₂)ₒ—,
—(CH₂)ₒX(CH₂)ₒ—,
—(CH₂)ₒX(CH₂)ₒO—,
—(CH₂)ₒX(CH₂)ₒNH—,
—C(O)(CH₂)ₒX(CH₂)ₒO—,
—C(O)(CH₂)ₒX(CH₂)ₒNH—,
(CH₂)₂C(O)NHXNHC(O)(CH₂)₂—,
—(CH₂)₂C(O)NHXC(O)NH(CH₂)₂—
—(CH₂)₂C(O)NHXOC(O)(CH₂)₂—,
—(CH₂)₂C(O)NHX(O)O(CH₂)₂—,
—(CH₂)₂C(O)OXOC(O)(CH₂)₂—, a complementary oligopeptide between 1 and 30 amino acids; a complementary oligonucleotide between 1 and 30 nucleic acid bases, X is alkyltriyl, heteroalkyltriyl, substituted heteroalkyltriyl; and o is an integer between 1 and 15.

17. The compound of claim 17, wherein R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

18. The compound of claim 1, wherein R¹ and R² are independently alkyl, alkenyl, aryl, or arylalkyl, R³, R⁴, R⁵ and R⁶ are independently —H, alkyl, alkenyl, aryl, arylalkyl or —SO₃H or a salt thereof, L₁, L₂, and L₃ are independently C₁-C₂₀ alkyl, phenyl, aryl, —NH—, —C(O)—, —(CH₂)ₙ—NH—C(O)—, —(CH₂)ₙ—C(O)—NH—, —(CH₂—CH₂—O)ₙ—, —(O—CH₂—CH(CH₃))ₙ—C(O)—NH—, or —(OCH₂—CH(CH₃))ₙ—; and each n is independently an integer from 1 to 25.

19. The compound of claim 19, wherein R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

20. The compound of claim 19, wherein R¹ and R² are independently —CH₃, —C₂H₅, or phenyl and R³, R⁴, R⁵ and R⁶ are independently —H, —CH₃, —C₂H₅, —C₃H₇, allyl, phenyl, benzyl or —SO₃H or a salt thereof.

21.-32. (canceled)
